(19) United States
(12) Patent Application Publication
(10) Pub. No.: US 2022/0356519 A1
(43) Pub. Date: Nov. 10, 2022

(54) SINGLE-MOLECULE SEEDING AND AMPLIFICATION ON A SURFACE

(71) Applicant: Pacific Biosciences of California, Inc., Menlo Park, CA (US)

(72) Inventors: Min-Jui Richard Shen, San Diego, CA (US); Kurt Patterson, San Diego, CA (US); Gary R. Abel, San Diego, CA (US); Hamed Hosseini Bay, San Diego, CA (US)

(21) Appl. No.: 17/740,058

(22) Filed: May 9, 2022

Related U.S. Application Data
(60) Provisional application No. 63/186,649, filed on May 10, 2021.
(60) Provisional application No. 63/283,877, filed on Nov. 29, 2021.

(51) Int. Cl. C12Q 1/6874

(52) U.S. Cl. CPC C12Q 1/6874; C12Q 1/6853

(57) ABSTRACT

Provided includes methods, compositions and systems for single molecule seeding and amplification on a flow cell. In some embodiments, nucleic acids are isothermally seeded and amplified on a flow cell comprising multiple binding areas (e.g., pads), resulting in an ensemble of substantially the same amplified molecules on each of the binding areas.

### CROSS-REFERENCE TO RELATED APPLICATIONS

This application claims the benefit of priority to U.S. Provisional Patent Application No. 63/186,649, filed May 10, 2021; and U.S. Provisional Patent Application No. 63/283,877, filed Nov. 29, 2021. The content of each of these related applications is incorporated herein by reference in its entirety.

### BACKGROUND

### Field

The present application generally relates to molecular biology and more specifically to amplification and sequencing of nucleic acids.

### Description of the Related Art

Rolling circle amplification (RCA) is an efficient method to amplify a circular template nucleic acid to produce long single stranded linear nucleic acid molecules that comprise concatenated copies of the template nucleic acid sequence. RCA has been used in many applications, such as nucleic acid sequencing.

### SUMMARY

Disclosed herein includes methods, systems and compositions of nucleic acid amplification. The method, in some embodiments, comprises: (a) providing a first circular DNA template comprising a first sequence, a second sequence, and a third sequence; (b) providing a surface comprising a plurality of binding areas, wherein each of the plurality of binding areas is attached thereto a plurality of oligonucleotide A and a plurality of oligonucleotide B, wherein the oligonucleotide A comprises a first capture sequence that is complementary to the first sequence and a second capture sequence that is complementary to the second sequence, and wherein the oligonucleotide B comprises the third sequence; and (c) contacting the first circular DNA template with the plurality of oligonucleotide A attached to a first binding area of the plurality of binding areas in the presence of a DNA polymerase to generate amplified single stranded concatemers of the first DNA template via rolling circle amplification (RCA), and contacting the single stranded oligonucleotide A primed concatemers to the plurality of oligonucleotide B to produce complementary concatemers of the first DNA template.

The method can, for example, further comprise providing a second circular DNA template comprising the first sequence, the second sequence, and the third sequence; and contacting the second circular DNA template with the plurality of oligonucleotide A being attached to a second binding area of the plurality of binding areas in the presence of the DNA polymerase to generate amplified single stranded concatemers of the second DNA template via RCA, and contacting the single stranded oligonucleotide A primed concatemers of the second DNA template to the plurality of oligonucleotide B to generate complementary concatemers of the second DNA template.

In some embodiments, contacting the first circular DNA template with the plurality of oligonucleotide A attached to a first binding area of the plurality of binding areas in the presence of a DNA polymerase and contacting the second circular DNA template with the plurality of oligonucleotide A being attached to a second binding area of the plurality of binding areas in the presence of the DNA polymerase occur simultaneously. In some embodiments, contacting the first circular DNA template with the plurality of oligonucleotide A attached to the first binding area of the plurality of binding areas in the presence of the DNA polymerase to generate amplified single stranded concatemers of the first DNA template via RCA comprises: hybridizing the first circular DNA template with an oligonucleotide A attached to the first binding area of the plurality of binding areas; and extending the oligonucleotide A bound to the first circular DNA template along the first circular DNA template by the DNA polymerase, whereby generating an amplified single stranded concatemer of the first DNA template.

In some embodiments, contacting the single stranded oligonucleotide A primed concatemers to the plurality of oligonucleotide B to produce complementary concatemers of the first DNA template comprises: hybridizing a single stranded oligonucleotide A primed concatemer with an oligonucleotide B attached to the first binding area of the plurality of binding areas; and extending the oligonucleotide B bound to the single stranded oligonucleotide A primed concatemer by the DNA polymerase, whereby generating a complementary concatemer of the first DNA template.

In some embodiments, extending of oligonucleotide A bound to the first circular DNA template along the first circular DNA template by the DNA polymerase and extending the oligonucleotide B bound to the single stranded oligonucleotide A primed concatemer occur simultaneously.

The method can, in some embodiments, comprise contacting complementary concatemers of the first DNA template with one or more oligonucleotide A of the plurality of oligonucleotide A attached to the first binding area to produce additional concatemers of the first DNA template. In some embodiments, the method comprises contacting complementary concatemers of the second DNA template with one or more oligonucleotide A of the plurality of oligonucleotide A attached to the second binding area to produce additional concatemers of the second DNA template.

The complementary concatemer of the first DNA template can be reverse complementary to the single stranded concatemer of the first DNA template. The first circular DNA template and the second circular DNA template can be provided in the same sample. In some embodiments, the first circular DNA template, the second circular DNA template, or both is a single-stranded DNA. In some embodiments, the first circular DNA template, the second circular DNA template, or both is circularized from a linear nucleic acid template. The surface can be a flow cell surface. In some embodiments, the RCA reaction is carried out within a flow cell.

The method can, in some embodiments, comprise terminating the RCA reaction by exhaustion of oligonucleotide A, oligonucleotide B, or both. In some embodiments, the method does not comprise terminating the RCA reaction by denaturing the DNA polymerase. In some embodiments, the method does not comprise terminating the RCA reaction by removing the DNA polymerase. In some embodiments, the RCA is performed at about 37° C. The DNA polymerase can be, for example, Phi29 DNA polymerase. In some embodiments, the first capture sequence is 5′ of the second capture sequence on the oligonucleotide A. In some embodiments, the second capture sequence is 5′ of the first capture sequence on the oligonucleotide A. The plurality of oligonucleotide A, the plurality of oligonucleotide B, or both can be covalently conjugated to the first binding area, the second binding area, or both. The plurality of oligonucleotide A, the plurality of oligonucleotide B, or both can be non-covalently attached to the first binding area, the second binding area, or both. In some embodiments, the plurality of oligonucleotide A and the plurality of oligonucleotide B are each attached at or near a 5′ end of the oligonucleotide A or oligonucleotide B. In some embodiments, the plurality of oligonucleotide A and the plurality of oligonucleotide B are not reverse complementary to one another. In some embodiments, a binding area of the plurality of binding areas is attached thereto a single oligonucleotide A, a single oligonucleotide B, or both. In some embodiments, a binding area of the plurality of binding areas is attached thereto at least 10,000 oligonucleotide A, at least 10,000 oligonucleotide B, or both.

A ratio of the plurality of oligonucleotide A and the plurality of oligonucleotide B attached to a binding area of the plurality of binding area can be about 100:1 to about 1:100. In some embodiments, the first binding area comprises a clonal population of the first DNA template. In some embodiments, the first binding area does not comprise the second DNA template. In some embodiments, the second binding area comprises a clonal population of the second DNA template. In some embodiments, the second binding area does not comprise the first DNA template. In some embodiments, at least 90% of the binding areas comprise clonal populations of no more than one nucleic acid template. In some embodiments, at least 90% of the binding areas comprise distinct template nucleic acids with respect to one another. The first sequence and the second sequence can be adjacent to one another. The third sequence can be adjacent to the first sequence or the second sequence.

The concatemers of the first DNA template and the complementary concatemers of the first DNA template can be attached to the first binding area of the plurality of binding areas via the plurality of oligonucleotide A and the plurality of oligonucleotide B attached to the first binding area. The concatemers of the second DNA template and the complementary concatemers of the second DNA template can be attached to the second binding area of the plurality of binding areas via the plurality of oligonucleotide A and the plurality of oligonucleotide B attached to the second binding area.

The surface can, for example, comprise about 10⁴ binding areas to about 10⁸ binding areas. In some embodiments, the surface comprises at least 10,000 ordered binding areas separated by disjunctions that are not predetermined and/or are randomly distributed. In some embodiments, one, one or more, or each, of the plurality of binding areas has a circular shape. The size of one, one or more, or each, of the plurality of binding areas can vary, for example about 10⁻⁹ m to about 10⁻⁴ m. In some embodiments, the size of the one, one or more, or each, of the plurality of binding areas is a width or a radius of the binding areas. In some embodiments, the surface is a planar surface.

Provided include methods, compositions and kits for amplifying nucleic acids. The method can comprise: (a) providing a first circular DNA template comprising a first sequence, a second sequence and a third sequence; (b) providing a surface comprising a plurality of binding areas, wherein each of the plurality of binding areas is attached thereto a plurality of oligonucleotide A and a plurality of oligonucleotide B, wherein the oligonucleotide A comprises a first capture sequence that is complementary to the first sequence and a second capture sequence that is complementary to the second sequence, and wherein the oligonucleotide B comprises the third sequence; and (c) contacting the first circular DNA template with the plurality of oligonucleotide A being attached to a first binding area of the plurality of binding areas in the presence of a DNA polymerase to generate amplified single stranded concatemers of the first DNA template via rolling circle amplification, and contacting the single stranded oligonucleotide A primed concatemers to the plurality of oligonucleotide B to generate complementary concatemers of the first DNA template.

### BRIEF DESCRIPTION OF THE DRAWINGS

FIG. 1 is a non-limiting schematic illustration showing generation of single-stranded DNA ring (ssDNA template). The ssDNA template comprises an adapter A having sequence 101 and sequence 103, and an adapter B having sequence 102.

FIG. 2 is a non-limiting schematic illustration showing an exemplary flow cell surface having multiple binding areas (e.g., pads), and the composition of individual pad having primers A and B conjugated thereto. Primer A has a sequence region complementary (or substantially complementary) to sequence 101 of the single-stranded DNA ring (ssDNA template) shown in FIG. 1, and another sequence region complementary (or substantially complementary) to sequence 102 of the single-stranded DNA ring (ssDNA template) shown in FIG. 1. Primer B has a sequence region having identical (or substantially identical) sequence to sequence 103 of the single-stranded DNA ring (ssDNA template) shown in FIG. 1.

FIG. 3 is a non-limiting schematic illustration showing capturing of single-stranded DNA template by primer A attached to a pad in the presence of a polymerase.

FIG. 4 is a non-limiting schematic illustration showing generation of DNA concatemers via rolling circle amplification.

FIG. 5 is a non-limiting schematic illustration showing annealing of primer B to a DNA concatemer.

FIGS. 6A-6B are non-limiting schematic illustrations showing: (1) generation of complementary DNA concatemer grown from priming using primer B via rolling circle amplification (FIG. 6A shows complementary concatemers grown from surface primer B; and (2) initiation of second strand priming with production of first strand (FIG. 6B shows that as more first strand is produced, more second strand priming is initiated from the surface).

FIG. 7 is a non-limiting schematic illustration showing formation of a cluster of DNA concatemers via rolling circle amplification. First strand primers can anneal to second strand scales for additional extension and surface primer consumption. Primer consumption can, in some embodiments, prevent additional library rings from annealing in the same pad.

FIG. 8 is a non-limiting schematic illustration showing amplification products of first and second strand concatemer DNA generated on multiple pads on a surface. Each pad can have first and second strand concatemer DNA.

FIG. 9A shows a non-limiting schematic illustration of producing a circular template nucleic acid hybridized to an immobilized primer.

FIG. 9B shows a non-limiting schematic illustration of extending an immobilized primer along a circular template nucleic acid via rolling circle amplification to produce amplified concatemers.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods, compositions and systems for using rolling circle amplification (RCA) to isothermally seed and amplify nucleic acids on a surface (e.g., a structured surface) comprising a plurality of binding areas (e.g., pads). Such seeding and amplification can result in a plurality of binding areas (e.g., pads) each containing an ensemble of essentially the same amplified molecules, and the ensemble of amplified molecules on the binding areas (e.g., pads) are different across the binding areas (e.g., pads) (that is, the ensemble of amplified molecules on a given binding area is different from the ensemble of amplified molecules on any other pad of the plurality of binding areas). The surface can be a flow cell surface. The surface can be planar or curved. Practice of the disclosure herein allows for simultaneous capture of a circular library component on a surface and amplification of that library component at a particular region of a structured surface. Often, capture and amplification occur at a rate faster than secondary capture, such that a particular region is seeded by no more than one library constituent. Consequently, the nucleic acid population seeded at that position is often a homogeneous amplification of a single original library constituent, so as to facilitate sequencing of the library constituent.

Rolling circle amplification produces a linear concatemeric nucleic acid molecule, which takes the form of a random coil, commonly referred to as a “picosphere.” A picosphere can be immobilized to a surface suitable for sequencing (e.g., via hybridizing to a universal capture oligonucleotide on the surface of a sequencing substrate). The universal capture oligonucleotide has a sequence that is unrelated to any specific target sequence of interest and thus can be used to capture any target sequences. The universal capture oligonucleotide can hybridize to the universal priming sequence in the picospheres. In some embodiments, the universal capture oligonucleotide is a barcode sequencing primer. In some embodiments, the picospheres is attached to the surface through ionic interactions, via covalent linkages, or mediated through binding of attached ligands (e.g., biotin and streptavidin). In some embodiments, one or several sequencing primers is hybridized to the picosphere before or after attachment to the surface for sequencing.

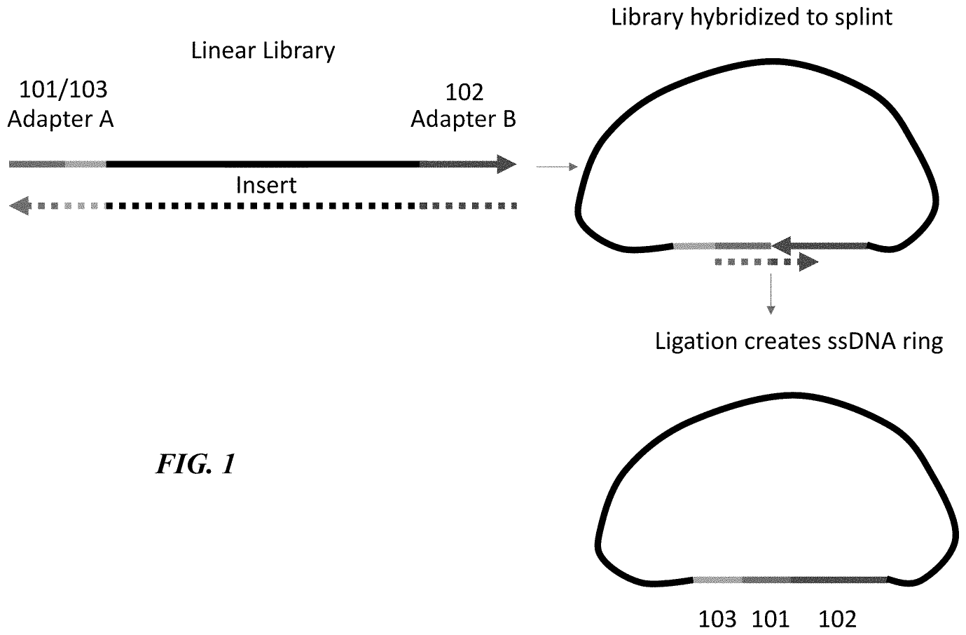

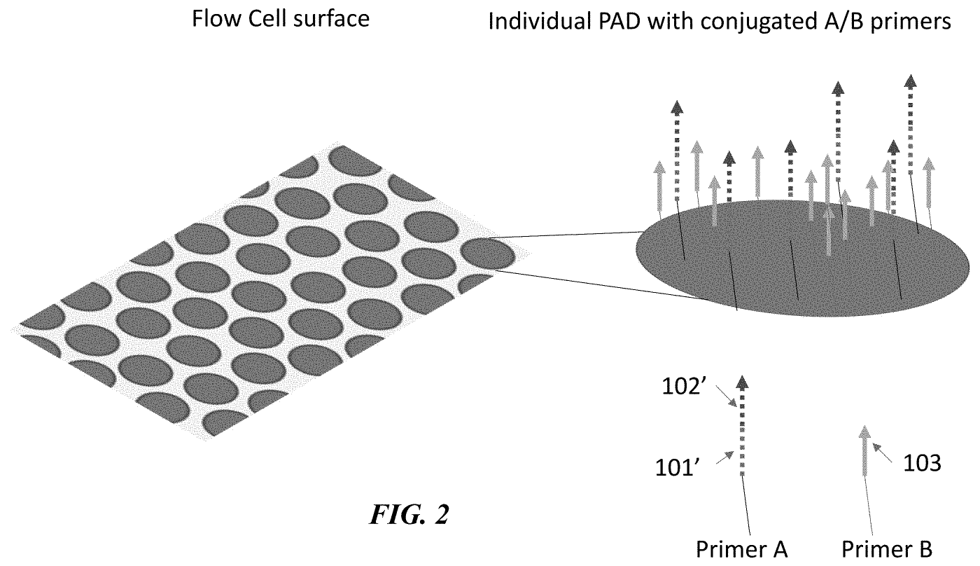

In the methods, compositions and systems disclosed herein, a surface (e.g., a structured surface) having a plurality of binding areas (e.g., pads) can be provided, wherein oligonucleotides A and B are immobilized on each of the plurality of binding areas. Oligonucleotides A comprises binding regions that are complementary and capable of capturing library elements, and oligonucleotide B comprises a binding region that is identical (or substantially identical) to a sequence of the library elements. For example, as shown in FIG. 1, adapter A and adapter B are at each end of a member nucleic acid of a linear library, the member nucleic acid can hybridize to splint and form ssDNA ring by ligation. Adapter A comprises sequence 101 and sequence 103, and adapter B comprises sequence 102. Upon ligation of adapter A to adapter B, a linear library constituent is converted to a circular molecule having an adapter A/adapter B ligated region that is reverse complementary to one set of oligonucleotides on the binding site, and locally identical in sequence to the second of the primers on the binding site. Consequently, the capturing oligonucleotide can serve to prime rolling circle amplification of the circularized library constituent, which in turn generates a linear concatemer reverse complementary to the original library constituent and having a segment that is reverse complementary to the second oligonucleotide on the surface. Shown in FIG. 2, a flow cell surface contains a plurality of binding areas (e.g., pads) and each of the binding areas has primers A and B attached (e.g., immobilized) thereon. Primers A and B each comprise sequence that spans the ligation event between adapters A and B of the original library constituent. Primers A and B are capable of functioning as primers in the subsequent reactions. Primer A comprises sequence 102′ that is complementary (or substantially complementary) to sequence 102 of adapter B, and sequence 101′ that is complementary (or substantially complementary) to sequence 101 of adapter A and located at 5′ of sequence 102′. Primer B comprises sequence 103 (or a sequence substantially the same as sequence 103). For primer A, sequence 101′ can be adjacent to sequence 102′ with no interval sequence in between, or the 3′ end of sequence 101′ can be one nucleotide, two nucleotides, three nucleotides, or more nucleotides away from 5′ end of sequence 102′. Because of the sequence complementarity, primer A can bind to the ssDNA ring template to generate DNA concatemers in the presence of DNA polymerase. Primer B can then bind to the concatemers generated by extension of the primer A bound to the circular template, generating reverse complement concatemers. However, primer A and primer B are not reverse complementary to one another, such that they do not form dimers on the surface.

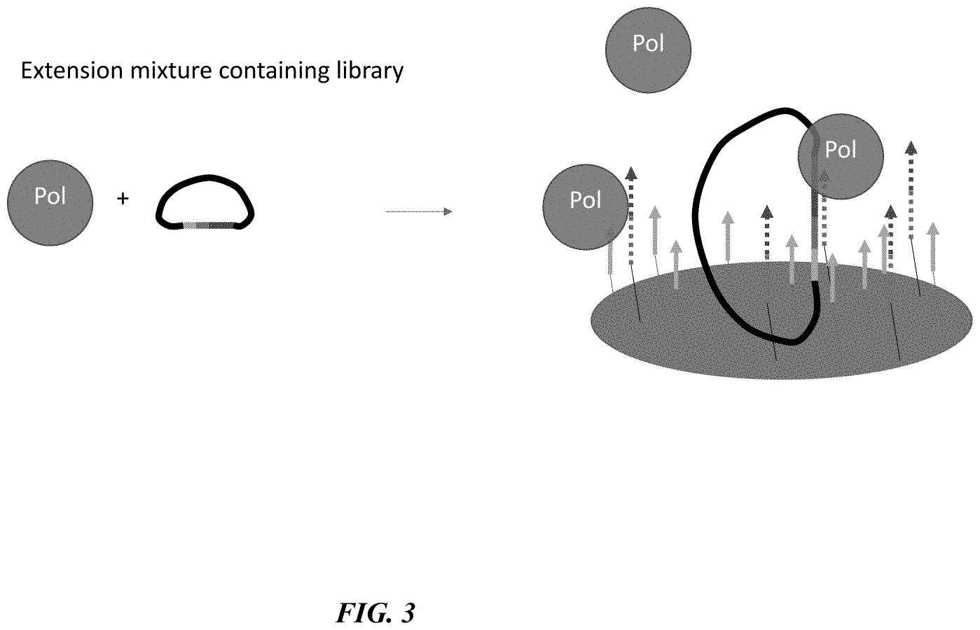

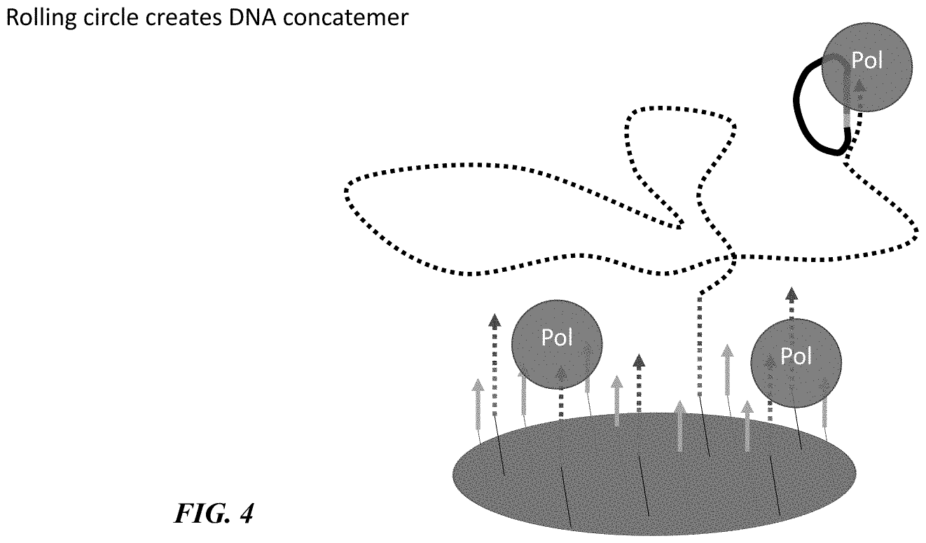

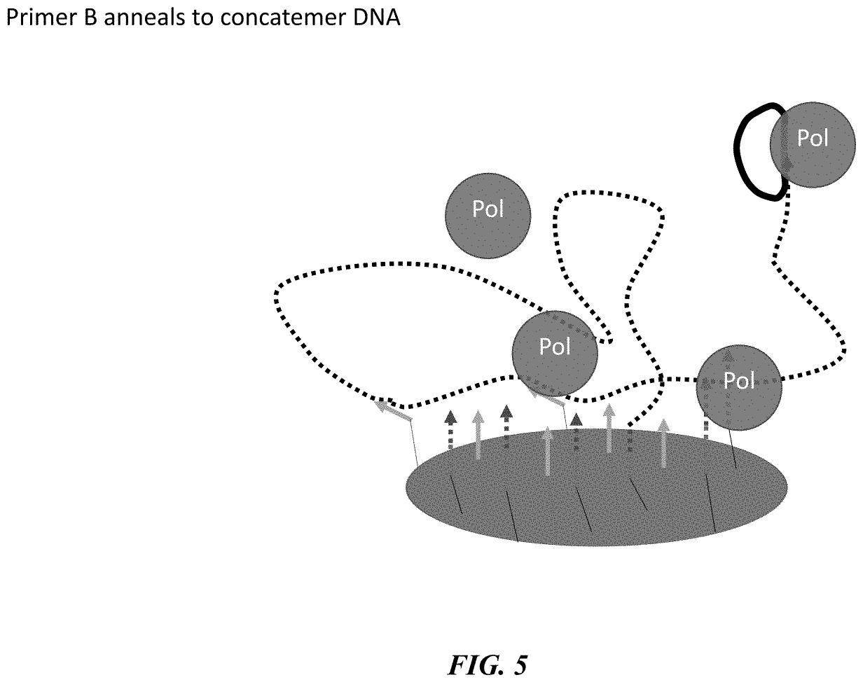

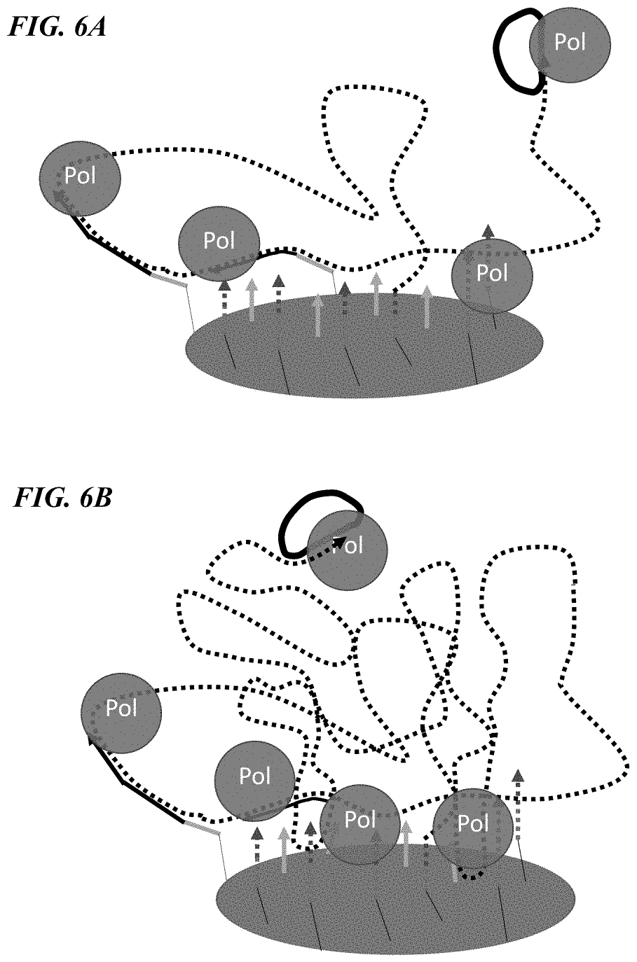

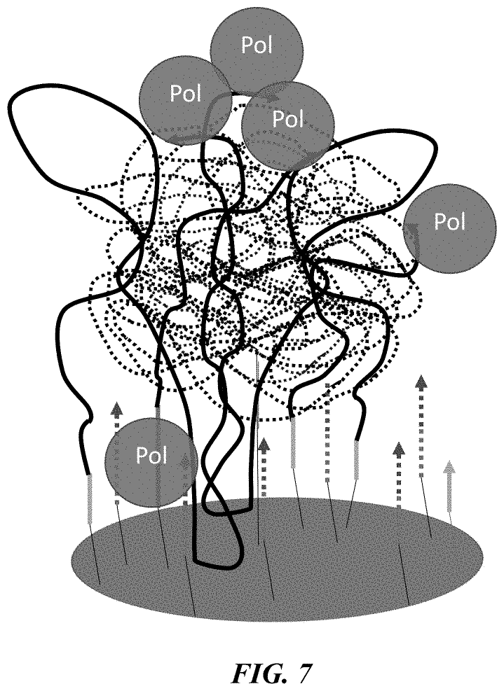

Extension mixture comprising library member nucleic acids in the form of ssDNA ring (also referred to as template ssDNA) and DNA polymerase can be formed, and provided to contact the pad on which oligonucleotides A and B are immobilized to perform nucleic acid extension and amplification (FIG. 3). Because of the sequence complementarity that oligonucleotides A and B have with adapter A and adapter B on the member nucleic acid of the library, respectively, oligonucleotides A and B can be used as capture oligonucleotides and amplification oligonucleotides. In some embodiments, a biochemical method allowing for simultaneous capture and amplification (e.g., rolling circle amplification (RCA) in the presence of, for example, phi29 polymerase, salts, nucleotides, buffer, or more) can be performed. For example, using oligonucleotide A as a primer, RCA can be carried out and generate concatemer copies of the template ssDNA (FIG. 4). Oligonucleotide B can then bind to the concatemer DNA and functions as a primer to allow RCA to generate complementary concatemers (FIGS. 5 and 6). However, as primer A and primer B are not reverse complementary to one another, they do not form primer dimers on the surface. As more first strand is generated, more second strand priming is initiated on the binding area (e.g., pad) to continue RCA (FIGS. 6A-6B). As shown in FIG. 7, first strand primers can anneal to second strand scales for additional extension and surface primer consumption. In some embodiments, it can be advantageous for primer consumption to prevent additional library rings from annealing in the same pad. As a result of the first strand priming RCA and the second priming RCA, the binding area (e.g., pad) will be associated with first and second strand concatemer DNA. The flow cell surface can have multiple binding areas (e.g., pads). The binding areas (e.g., pads) can be in a structured configuration on the flow cell surface, or in some embodiments, the pads are in configurations that are not structured and/or pre-determined.

The method can include: (a) providing a first circular DNA template comprising a first sequence, a second sequence and a third sequence; (b) providing a surface comprising a plurality of binding areas, wherein each of the plurality of binding areas is attached thereto a plurality of oligonucleotide A and a plurality of oligonucleotide B, wherein the oligonucleotide A comprises a first capture sequence that is complementary to the first sequence and a second capture sequence that is complementary to the second sequence, and wherein the oligonucleotide B comprises the third sequence; and (c) contacting the first circular DNA template with the plurality of oligonucleotide A and the plurality of oligonucleotide B being attached to a first binding area of the plurality of binding areas in the presence of a DNA polymerase to generate amplified concatemers of the first DNA template and complementary concatemers of the first DNA template. In some embodiments, some methods begin with a linear library constituent having adapters A and B at its end, such that contact with Primer A leads to positioning the linear library constituent for circularization upon contacting with a ligase. The generation of amplified concatemers of the first DNA template and complementary concatemers of the first DNA template can be via a rolling cycle amplification (RCA) reaction, for example a RCA reaction carried out in an isothermal condition at about, or at a temperature in the range of 25° C. to 65° C., for example 37° C. The complementary concatemers generated thereby comprise a region complementary to primer B, such that primer B can prime the synthesis of reverse-complementary concatemers or reverse complementary strands to the primer A primer concatemer strands.

The method, in some embodiments, includes: (a) providing a plurality of circular DNA template each comprising a first sequence, a second sequence, and a third sequence; (b) providing a surface comprising a plurality of binding areas, wherein each of the plurality of binding areas is attached thereto a plurality of oligonucleotide A and a plurality of oligonucleotide B, wherein the oligonucleotide A comprises a first capture sequence that is complementary to the first sequence and a second capture sequence that is complementary to the second sequence, and wherein the oligonucleotide B comprises the third sequence; and (c) hybridizing each of the plurality of circular DNA templates to the plurality of oligonucleotide A and the plurality of oligonucleotide B being attached to a binding area of the plurality of binding areas, respectively, in the presence of a DNA polymerase to generate amplified concatemers of the DNA template and complementary concatemers of the DNA template via rolling cycle amplification (RCA). Such seeding and amplification method can result in a plurality of binding areas each containing an ensemble of essentially the same amplified molecule, and the ensemble of amplified molecules on the binding areas are different across the binding areas (that is, the ensemble of amplified molecules on a given binding area is different from the ensemble of amplified molecules on any other pad of the plurality of binding areas).

### Definitions

Unless defined otherwise, technical and scientific terms used herein have the same meaning as commonly understood by one of ordinary skill in the art to which the present disclosure belongs. See e.g., Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, the term “immobilized,” when used in reference to a molecule, refers to direct or indirect, covalent or non-covalent attachment of the molecule to a surface such as a surface of a solid support. In some configurations, covalent attachment is preferred, but generally all that is required is that the molecules (e.g., nucleic acids) remain immobilized or attached to the surface under the conditions in which surface retention is intended.

As used herein, the term “nucleotide” can be used to refer to a native nucleotide or analog thereof. Examples include, but are not limited to, nucleotide triphosphates (NTPs) such as ribonucleotide triphosphates (rNTPs), deoxyribonucleotide triphosphates (dNTPs), or non-natural analogs thereof such as dideoxyribonucleotide triphosphates (ddNTPs) or reversibly terminated nucleotide triphosphates (rtNTPs).

As used herein, the terms “complementarity” or “complementary” mean that a nucleic acid can form hydrogen bond(s) with another nucleic acid based on traditional Watson-Crick base paring rule. Complementarity can be complete or partial. Complete complementarity indicates that each and every nucleic acid base of one strand is capable of forming hydrogen bonds according to Watson-Crick canonical base pairing with a corresponding base in another, antiparallel nucleic acid sequence. Partial complementarity indicates that only a percentage of the contiguous residues of a nucleic acid sequence can form Watson-Crick base pairing with the same number of contiguous residues in another, antiparallel nucleic acid sequence. “Substantially complementary” refers to a percentage of complementary of about, at least, or at least about 70%, 80%, 90%, 100% or a number or a range between any two of these values.

As used herein, the term “polymerase” can be used to refer to a nucleic acid synthesizing enzyme, including but not limited to, DNA polymerase, RNA polymerase, reverse transcriptase, primase and transferase. Typically, the polymerase has one or more active sites at which nucleotide binding and/or catalysis of nucleotide polymerization may occur. The polymerase can catalyze the polymerization of nucleotides to the 3′ end of the first strand of the double stranded nucleic acid molecule. For example, a polymerase catalyzes the addition of a next correct nucleotide to the 3′ oxygen moiety of the first strand of the double stranded nucleic acid molecule via a phosphodiester bond, thereby covalently incorporating the nucleotide to the first strand of the double stranded nucleic acid molecule. In some embodiments, a polymerase need not be capable of nucleotide incorporation under one or more conditions used in a method set forth herein. For example, a mutant polymerase can be capable of forming a ternary complex but incapable of catalyzing nucleotide incorporation.

As used herein, the term “primer” refers to a nucleic acid having a sequence that binds to a nucleic acid at or near a template sequence. Generally, the primer binds in a configuration that allows replication of the template, for example, via polymerase extension of the primer. The primer can be a first portion of a nucleic acid molecule that binds to a second portion of the nucleic acid molecule, the first portion being a primer sequence and the second portion being a primer binding sequence (e.g., a hairpin primer). In some embodiments, the primer is a first nucleic acid molecule that binds to a second nucleic acid molecule having the template sequence. A primer can consist of DNA, RNA or analogs thereof. A primer can have an extendible 3′ end or a 3′ end that is blocked from primer extension.

As used herein, a “vessel” is a container that functions to isolate one chemical process (e.g., a binding event; an incorporation reaction; etc.) from another, or to provide a space in which a chemical process can take place. Examples of vessels useful in connection with the disclosed technique include, but are not limited to, flow cells, wells of a multi-well plate; microscope slides; tubes (e.g., capillary tubes); droplets, vesicles, test tubes, trays, centrifuge tubes, features in an array, tubing, channels in a substrate etc.

As used herein, the term “circular,” when used in reference to a nucleic acid strand, means that the strand has no terminus (that is, the strand lacks a 3′ end and a 5′ end). Accordingly, the 3′ oxygen and the 5′ phosphate moieties of every nucleotide monomer in a circular strand is covalently attached to an adjacent nucleotide monomer in the strand. A circular DNA strand can serve as a template for producing a concatemeric amplicon via rolling circle amplification (RCA), wherein each sequence unit of the concatemeric amplicon is the reverse complement of the circular nucleic acid strand. A circular nucleic acid can be double stranded. One or both strands in a double stranded nucleic acid can lack a 3′ end and a 5′ end. One strand in a double stranded nucleic acid can have a gap (absence of at least one nucleotide monomer relative to the other strand) or nick (absence of a phosphodiester bond between two nucleotide monomers), so long as the other strand is circular.

As used herein, the term “concatemer,” when used in reference to a nucleic acid molecule, means a continuous nucleic acid molecule that contains multiple copies of a common sequence linked in series. Similarly, the term “concatemer,” when used in reference to a nucleotide sequence, means a continuous nucleotide sequence that contains multiple copies of a common sequence in series. Each copy of the sequence can be referred to as a “sequence unit” of the concatemer. A sequence unit can have a length of at least 10 bases, 50 bases, 100 bases, 250 bases, 500 bases or more. A concatemer can include at least 2, 5, 10, 50, 100 or more sequence units. A sequence unit can include subregions having any of a variety of functions such as a primer binding region, target sequence region, tag region, unique molecular identifier (UMI), or the like.

As used herein, a “flow cell” is a reaction chamber that includes one or more channels that direct fluid in a predetermined manner to conduct a desired reaction. The flow cell can be coupled to a detector such that a reaction occurring in the reaction chamber can be observed. For example, a flow cell can contain primed template nucleic acid molecules, for example, tethered to a solid support, to which nucleotides and ancillary reagents are iteratively applied and washed away. The flow cell can include a transparent material that permits the sample to be imaged after a desired reaction occurs. For example, a flow cell can include a glass slide containing small fluidic channels, through which polymerases, dNTPs and buffers can be pumped. The glass inside the channels is decorated with one or more primed template nucleic acid molecules to be sequenced. An external imaging system can be positioned to detect the molecules on the surface of the glass. Reagent exchange in a flow cell is accomplished by pumping, drawing, or otherwise “flowing” different liquid reagents through the flow cell. Exemplary flow cells, methods for their manufacture and methods for their use are described in U.S. Patent Application Publications US2010/0111768 or US2012/0270305; or WO 05/065814, each of which is incorporated by reference herein.

As used herein, the term “cluster,” when used in reference to nucleic acids, refers to a population of nucleic acids that is attached to a solid support, for example, at a binding area in an array of binding areas on the solid support.

As used herein, the term “clonal population” refers to a population of nucleic acids that is homogeneous with respect to a particular nucleic acid sequence. The homogenous sequence is typically at least 10 nucleotides long, but can be even longer including for example, at least 50, 100, 500, 1000 or 2500 nucleotides long. A clonal population can be derived from a single template nucleic acid. A clonal population can include at least 2, 10, 100, 500, or 1000 copies of a particular nucleic acid sequence. The copies can be present in a single nucleic acid molecule, for example, as a concatemer, or the copies can be present on separate nucleic acid molecules (e.g., separate concatemers). Typically, all the nucleic acids in a cluster will have the same nucleotide sequence. It will be understood that a negligible number of contaminant nucleic acids or mutations (e.g., due to amplification artifacts) can occur in a cluster without departing from apparent clonality. A cluster can be at least 80%, 85%, 90%, 95%, or 99% clonal. In some embodiments, a cluster can be 100% clonal.

### Rolling Circle Amplification

Provided herein include methods, systems and compositions for using rolling circle amplification to seed and amplify nucleic acids on a surface. Generally, a RCA reaction involves a polymerase extending a primer that is annealed to a circular template such that multiple laps of the polymerase around the circular template produces a concatemeric single stranded DNA that contains multiple tandem repeats, each of the repeats being complementary to the circular template.

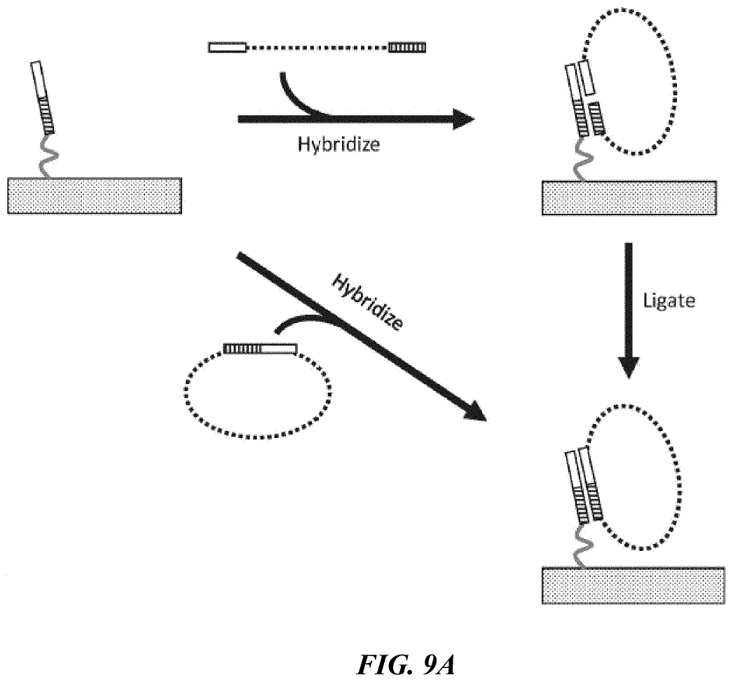

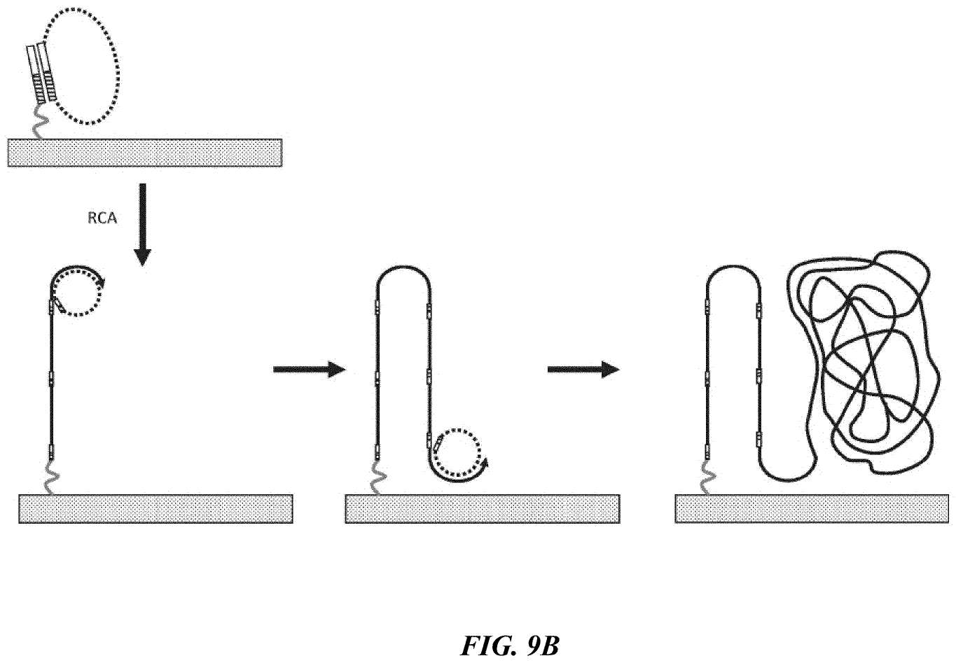

FIGS. 9A-B provide a non-limiting schematic illustration of performing a rolling circle amplification of nucleic acids. As shown in FIG. 9A, a nucleic acid primer (indicated by the open and lined rectangles) (e.g., oligonucleotide A or primer A in FIG. 2) is attached to a solid support (indicated by the dotted rectangle) via a linker (indicated by the grey line). The primer can be used to capture a target nucleic acid of a template nucleic acid via a primer binding site in the template nucleic acid that is complementary to the primer. For example, the primer can have a first capture sequence (e.g., indicated by the open rectangle) that is complementary to a first primer binding region of the template nucleic acid (e.g., indicated by the open rectangle) and a second capture sequence (e.g., indicated by the lined rectangle) that is complementary to a second primer binding region of the template nucleic acid (e.g., indicated by the line rectangle).

In an exemplary configuration shown in FIG. 9A, the immobilized primer can hybridize to portions of the primer binding site that are present at opposite ends of a target sequence (the target sequence being indicated by a dotted line and the flanking primer binding site regions being indicated by open and lined rectangles, respectively). The immobilized primer thus functions as a splint that brings together the two ends of the template nucleic acid. The two ends can be ligated, for example, by a ligase, while hybridized to a splint nucleic acid to form a circular version of the template nucleic acid. In another configuration shown in FIG. 9A, a template nucleic acid is circularized prior to being hybridized to the immobilized primer on the solid support. Accordingly, the template sequence can be a linear nucleic acid or a circular nucleic acid.

FIG. 9B provides a non-limiting schematic illustration of a single-stranded concatemer being produced via rolling circle amplification of a primed circular template that is hybridized to an immobilized primer. The primer is immobilized in a way that the 3′ end is available for polymerase extension (e.g., the primer can be attached at or near its 5′ end). The product of the first sub-step is shown as having progressed to a point that two copies of the circular template (two sequence units) have already been produced and the circular template is hybridized to a portion of a third copy (third sequence unit) that is being replicated. Each of the sequence units includes a region that is complementary to the target sequence (indicated by the solid black line) and a region that is complementary to the primer (indicated by the open and lined rectangles). The product of the second sub-step has progressed to the point of having produced nearly six copies of the circular template. FIG. 9B shows a product of the RCA reaction after the circular template is absent (e.g., has been removed) in the third sub-step. Two regions of the final product are shown for illustrative purposes: a region where the sequence units are delineated (indicative of the concatemeric primary structure of the amplified strand) and a region where the number and conformation of the sequence units is not specified (indicative of the dynamic and variable secondary structure for the cluster as a whole).

An RCA reaction can be terminated by denaturing the polymerase, for example, by heating the sample at 60° C., 65° C., 70° C., 75° C., 80° C., or higher. An RCA reaction can also be terminated by removing one or more components of RCA, such as the polymerase and/or the dNTPs. In some embodiments, the RCA reaction can be terminated by exhaustion of primers. Components of RCA can be removed by, for example, washing with a washing reagent.

### Seeding and Amplification of Nucleic Acids on a Surface

Disclosed herein includes a method of nucleic acid amplification, and in particular a method to seed and amplify nucleic acids on a surface comprising a plurality of binding areas. In some embodiments, the method can comprise providing a nucleic acid template comprising one or more primer binding regions (e.g., a first sequence, a second sequence and a third sequence). The nucleic acid template can be a circular nucleic acid or a linear nucleic acid circularized to form a circular nucleic acid. The one or more primer binding regions can be adjacent to one another (such as in a circular template nucleic acid). The one or more primer binding regions can be located at the opposite ends of a linear template nucleic acid. In some embodiments, the primer binding regions are the regions originally used to circularize the linear nucleic acid library constituent (see e.g., FIG. 1). The nucleic acid template also comprises a target region containing a target nucleic acid that is the object of amplification. The term “providing” as used herein refers to the preparation and delivery of one or more components (e.g., the nucleic acid template) to a vessel where the RCA reaction takes place, such as a surface of a flow cell comprising a plurality of binding areas.

The method also comprises contacting the nucleic acid template with a plurality of oligonucleotide A and a plurality of oligonucleotide B attached (e.g., covalently or non-covalently) to a binding area of the plurality of binding areas in the presence of a polymerase (e.g., DNA polymerase) to generate a plurality of amplified single-stranded concatemers of the nucleic acid template via rolling circle amplification. The plurality of amplified single-stranded concatemers generated from the RCA herein described each comprises multiple copies of the nucleic acid template or a reverse complement thereof.

In some embodiments, contacting the nucleic acid template with the plurality of oligonucleotide A and the plurality of oligonucleotide B attached to a binding area comprises (i) contacting the nucleic acid template with the plurality of oligonucleotide A to generate single-stranded, oligonucleotide A primed concatemers (or first strand concatemers) of the nucleic acid template via rolling circle amplification and (ii) contacting the single-stranded, oligonucleotide A primed concatemers to the plurality of oligonucleotide B to generate single-stranded, oligonucleotide B primed concatemers (or second strand concatemers) via rolling circle amplification, the oligonucleotide B primed concatemers being reverse complementary to the oligonucleotide A primed concatemers. As used herein, “oligonucleotide A primed concatemers” or “first strand concatemers” refer to concatemers generated from extending oligonucleotide A along, for example, the template nucleic acids. Similarly, “oligonucleotide B primed concatemers” or “second strand concatemers” refer to concatemers generated from extending oligonucleotide B along, for example, the first strand concatemers.

In some embodiments, the step of generating the first strand concatemers using the plurality of oligonucleotide A and the step of generating the second strand concatemers using the plurality of oligonucleotide B can occur sequentially or simultaneously. For example, in some instances, oligonucleotide B can prime and be extended along the first strand concatemers while the first strand concatemers are still being extended via the RCA reaction. In some other instances, oligonucleotide B can prime and be extended along the first strand concatemers after the extension of the first strand concatemers is terminated. As more first strand concatemers are produced, more second strand priming is initiated from the surface.

The generated first strand and second strand concatemers can subsequently initiate additional rounds of replication by annealing to fresh immobilized oligonucleotides A and B (or unused primers) on the surface, so as to form a series of immobilized concatemeric strands each containing multiple sequence units, each sequence unit being either substantially complementary or substantially identical to the template nucleic acid. For example, the second strand concatemers can contact and hybridize to one or more fresh oligonucleotide A, thus facilitating new rounds of amplification to form additional first strand concatemers. The additional first strand concatemers can contact and hybridize to one or more fresh oligonucleotide B, thus facilitating new rounds of amplification to form additional second strand concatemers. Additional rounds of priming and amplification allow primers to be consumed and a multitude of concatemeric strands to be synthesized in a short period of time. Primer consumption can prevent additional template nucleic acids from annealing in the same binding area. In some embodiments, the template sequences can be subjected to about, at least, at least about, at most, or at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, or a number or a range between any two of these values, rounds of replication via rolling circle amplification.

The seeding and amplification therein described result in a binding area having tethered thereto a plurality of concatemeric nucleic acid molecules, where a concatemeric strand can be partially hybridized to multiple complementary concatemeric strands. The concatemeric strands and complementary concatemeric strands can be subjected to next generation sequencing. The concatemeric strands and complementary concatemeric strands can be sequenced when they are partially hybridized to one another. Alternatively or additionally, the concatemeric strands and the complementary concatemeric strands can be separated by, for example, heat denaturation such that the strands are not partially hybridized.

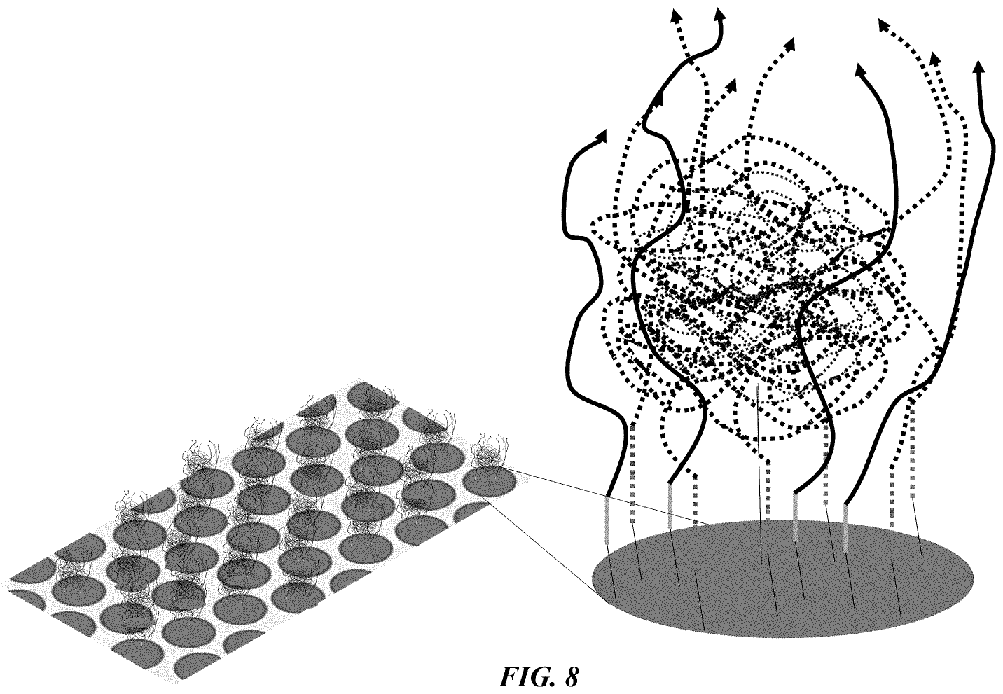

The multiple rounds of amplification herein described can produce a nucleic acid cluster comprising a population of nucleic acids (e.g., concatemeric strands) attached to a surface, for example, at a binding area of the surface (see FIG. 8 for a non-limiting example). In some embodiments, a cluster at a binding area of the surface is homogeneous with respect to a particular nucleic acid sequence such that about, at least or at least about 80%, 90%, 95% or 99% of the amplified nucleic acid in the cluster contain the same target sequence. Therefore, the seeding and amplification method herein described can result in an ensemble of substantially the same amplified molecules on each of the binding areas.

In some embodiments herein described, a vessel where the template nucleic acids and/or the concatemers are being contacted with the immobilized oligonucleotides is a flow cell. Accordingly, in some embodiments, the contacting steps can be facilitated by the use of a flow cell. Flowing liquid reagents (e.g., extension mixture) through a flow cell can permit reagent mixing and exchange. For example, contacting the nucleic acid template with the plurality of immobilized oligonucleotides can comprise flowing the extension mixture comprising a polymerase, a dNTP mix, a template nucleic acid through a flow cell having a surface comprising a plurality of binding areas. The extension mixture can also include auxiliary reagents necessary for carrying out a RCA reaction such as salts, buffers, small molecules, co-factors, metals and ions as will be apparent to a skilled person.

The extension mixture can be incubated with the immobilized primers at any temperature conducive to the polymerase activity. In some embodiments, the RCA reactions herein described do not require thermocycling, and can be performed at a substantially isothermal reaction temperature, for example, a temperature that does not vary more than by about 2-3° C. above or below a given temperature. The reaction temperature can be between 20° C. and 70° C., for example 20° C., 25° C., 30° C., 35° C., 37° C., 40° C., 45° C., 50° C., 55° C., 60° C., 65° C., 70° C., or a number or a range between any two of these values. In some embodiments, the reaction temperature is between 20° C. and 60° C. In some embodiments, the reaction temperature is between 20° C. and 50° C. In some embodiments, the reaction temperature is about 30° C. (e.g., about 37° C.).

The extension reaction primed by oligonucleotides A and B can be terminated by the exhaustion of oligonucleotides A and B on a binding area. In some embodiments, the extension reaction can be terminated, for example, by introduction of a 3′ blocking nucleotide that can inhibit or prevent the 3′ oxygen of the nucleotide from forming a covalent linkage to a next nucleotide during a nucleic acid polymerization reaction. In some embodiments, the extension reaction is not terminated by denaturing the polymerase (e.g., DNA polymerase), by removing the polymerase, or by any chemical inactivation of the polymerase.

### Generation of the First Strand Concatemers

In some embodiment, the step (i) of contacting the nucleic acid template with the plurality of oligonucleotide A to generate single-stranded, oligonucleotide A primed concatemers of the nucleic acid template can comprise hybridizing the nucleic acid template with the plurality of oligonucleotide A (e.g. through the complementary base pairing between the capture sequence of oligonucleotide A and the first and second sequence of the nucleic acid template) (e.g., in FIG. 3). Oligonucleotide A is then subjected to an extension reaction, so as to have added at its 3′ end multiple monomer units of the nucleic acid template, via rolling circle amplification. The result is a concatemer of multimers of the original nucleic acid template tethered to the surface via oligonucleotide A as seen in FIG. 4. Accordingly, the method comprises extending the plurality of oligonucleotide A bound to the nucleic acid template to generate the oligonucleotide A primed concatemers (or the first strand concatemers) of the nucleic acid template.

One or more oligonucleotide A primed concatemers (or first strand concatemers) can be produced in a non-limiting exemplary embodiment set forth in FIGS. 3-4. A plurality of oligonucleotide A (indicated by dotted arrows) and B (indicated by solid gray arrows) are immobilized via linkers (indicated by black lines and gray lines) on a binding area (indicated by a blue oval). As shown in FIG. 3, a nucleic acid template is captured by oligonucleotide A via the complementary base pairing between the primer binding sequence of the nucleic acid template (e.g., the first sequence and the second sequence indicated as solid green and red lines) and the corresponding capture sequence in oligonucleotide A (indicated by dotted green and red arrows). The bound oligonucleotide A is then extended in a RCA reaction by a polymerase, such as a polymerase with strand displacement activity, along the template nucleic acid to produce a first strand concatemer (shown as the dotted black line partially annealed to the template nucleic acid in FIG. 4). The generated first strand concatemer remains attached to the surface via primer A. Primer A can be used as a capture primer and an amplification primer. One or more nucleic acid templates can be captured and amplified simultaneously, generating a plurality of first strand concatemers of the nucleic acid templates. In some embodiments, one or at most one template nucleic acid is captured and amplified at a binding area of the plurality of binding areas, generating one or at most one first strand concatemer at the binding area. The number of oligonucleotide A and B immobilized on the binding area and the number of strands generated shown in the figures are for illustration only and not intended to be limiting.

In some embodiments, the template nucleic acid does not hybridize with oligonucleotide B because the capture sequence in oligonucleotide B is substantially identical to the third sequence of the nucleic acid template and not reverse complementary to the first and the second sequence of the nucleic acid template.

Each of the sequence units in a first strand concatemer includes a region that is complementary to the target sequence and a region that is complementary to the primer binding sequences (e.g., the first sequence, the second sequence, and the third sequence). Accordingly, each of the sequence units in the first strand concatemer contains a segment that is reverse complementary to oligonucleotide B or a portion thereof.

### Generation of the Second Strand Concatemers

Upon the generation of the first strand concatemers of the nucleic acid template, the first strand concatemers are then used as templates for generating the second strand concatemers of the nucleic acid template, primed by oligonucleotide B that can anneal to a portion of a primer binding region of the first strand concatemer, for example, the portion of the primer binding region complementary to the sequence of oligonucleotide B.

Similarly, the step of (ii) contacting the single-stranded, oligonucleotide A primed concatemers with the plurality of oligonucleotide B to generate single-stranded, oligonucleotide B primed concatemers can comprise hybridizing the oligonucleotide A primed concatemers (or the first strand concatemers) with the plurality of oligonucleotide B and extending the plurality of oligonucleotide B bound to the oligonucleotide A primed concatemers to generate the oligonucleotide B primed concatemers (or the second strand concatemers) of the nucleic acid template.

One or more oligonucleotide B primed concatemers (or second strand concatemers) can be produced in a non-limiting exemplary embodiment set forth in FIGS. 5-6 using the plurality of oligonucleotide B attached to the binding area. As shown in FIG. 5, oligonucleotide B (indicated by a gray arrow) can prime a first strand concatemer (indicated by the dotted black line) by hybridizing to a portion of the first strand concatemer and being extended via RCA by a polymerase along the first strand concatemer to generate a second strand concatemer. The second strand concatemers generated (indicated by solid black lines in the figures) remain attached to the surface via oligonucleotide B. Multiple first strand concatemers each can be primed by one or more oligonucleotide B simultaneously, generating a plurality of second strand concatemers of the nucleic acid templates immobilized to the surface.

In some embodiments, one or more second strand concatemers can be produced from a single first strand concatemer. For example, one or more oligonucleotide B can anneal to a first strand concatemer (e.g., different sequence units of the first strand concatemer) to produce a nucleic acid cluster having the first strand concatemer hybridized to the one or more extending oligonucleotide B, thereby generating one or more second strand concatemers that complement at least a portion of the first strand concatemer. The one or more second strand concatemers generated from a same first strand concatemer can have a variety of length (e.g., various number of sequence units) and annealing patterns. In one example shown in FIG. 6A, two oligonucleotide B hybridize to two distinct sequence units of a first strand concatemer and extend in a RCA reaction to produce two second strand concatemers (shown as solid black lines annealed, at least partially, to the first strand). The number of oligonucleotide A and B immobilized on the binding area and the number of first and second strands generated shown in the figures are for illustration only and not intended to be limiting.

### Multiplexed Seeding and Amplification

The methods described herein can be carried out in a multiplex format such that multiple different template nucleic acids can be seeded and amplified in parallel on discrete binding areas using the steps set forth herein (see e.g., FIG. 8). Each binding area of a plurality of binding areas can generate a plurality of concatemer strands each containing multiple copies of a particular target sequence or a reverse complement thereof (see e.g., FIG. 8). For example, a plurality of nucleic acid templates containing different target sequences (e.g., a sequencing library) can be distributed over the plurality of binding areas of a surface such that different library constituents are captured and amplified in discrete binding areas via rolling circle amplification. Each binding area can contain a colony having the first strand and second strand concatemers of a particular target nucleic acid tethered to the binding area via oligonucleotides A and B. In some embodiments, the method herein disclosed can be configured to seed and amplify about, at least, at least about, at most, or at most about 2, 10, 100, 1×10³, 1×10⁴, 1×10⁵, 1×10⁶, 1×10⁹, or more different nucleic acids in parallel, thus providing cost savings, time savings, and uniformity of conditions. As such, the number of binding areas on a surface can be in a range exemplified here for different nucleic acids.

For example, in some embodiments, a second nucleic acid template is provided to a second binding area that is different from a first binding area on which a first nucleic acid template is captured and amplified. The second nucleic acid template can contain a target nucleic acid having a sequence different from the target nucleic acid in the first nucleic acid template.

Accordingly, in some embodiments, the method can comprise providing a second nucleic acid template (e.g., a second circular DNA template) comprising the first sequence, the second sequence, and the third sequence and contacting the second nucleic acid template with the plurality of oligonucleotide A and oligonucleotide B attached to a second binding area of the plurality of binding areas in the presence of a polymerase to generated amplified single-stranded concatemers of the second nucleic acid template via rolling circle amplification. The plurality of amplified single-stranded concatemers generated from the RCA herein described each comprises multiple copies of the second nucleic acid template or a reverse complement thereof.

In some embodiments, contacting the second nucleic acid template with the plurality of oligonucleotide A and the plurality of oligonucleotide B attached to the second binding area comprises (i) contacting the second nucleic acid template with the plurality of oligonucleotide A to generate single-stranded, oligonucleotide A primed concatemers (or first strand concatemers) of the second nucleic acid template via rolling circle amplification and (ii) contacting the single-stranded, oligonucleotide A primed concatemers to the plurality of oligonucleotide B to generate single-stranded, oligonucleotide B primed concatemers (or second strand concatemers) of the second nucleic acid template via rolling circle amplification, the oligonucleotide B primed concatemers being reverse complementary to the oligonucleotide A primed concatemers.

Similar to the amplification of the first nucleic acid template on the first binding area, the step of generating the first strand concatemers of the second nucleic acid template using the plurality of oligonucleotide A and the step of generating the second strand concatemers of the second nucleic acid template using the plurality of oligonucleotide B can occur sequentially or simultaneously. For example, in some instances, oligonucleotide B can prime the first strand concatemers of the second nucleic acid template while the first strand concatemers are still being extended via the RCA reaction. In some other instances, oligonucleotide B can prime the first strand concatemers after the extension of the first strand concatemers is terminated.

In some embodiments, the first binding area can comprise a clonal population of the first nucleic acid template and the second binding area can comprise a clonal population of the second nucleic acid template. For example, in some embodiments, the first binding area does not comprise the second nucleic acid template and the second binding area does not comprise the first nucleic acid template.

In some embodiments, a plurality of nucleic acid templates can be provided to a surface comprising a plurality of binding areas. Accordingly, in some embodiments, the method can comprise (a) providing a plurality of nucleic acid templates (e.g., circular DNA templates) each comprising a first sequence, a second sequence, and a third sequence; (b) providing a surface comprising a plurality of binding areas, wherein each of the plurality of binding areas is attached thereto a plurality of oligonucleotide A and a plurality of oligonucleotide B, wherein the oligonucleotide A comprises a first capture sequence that is complementary to the first sequence and a second capture sequence that is complementary to the second sequence, and wherein the oligonucleotide B comprises the third sequence; and (c) hybridizing each of the plurality of circular DNA templates to the plurality of oligonucleotide A and the plurality of oligonucleotide B being attached to a binding area of the plurality of binding areas, respectively, in the presence of a DNA polymerase to generate amplified concatemers of the DNA template and complementary concatemers of the DNA template via rolling cycle amplification (RCA). Amplified concatemers can be independently generated at each binding area where a different template nucleic acid is amplified.

The multiplexed seeding and amplification can result in a plurality of binding areas each containing a nucleic acid cluster attached to a binding area. The nucleic acid cluster generated at or on a binding area can comprise no more than one nucleic acid template. For example, the percentage of the plurality of binding areas each comprising no more than one nucleic acid template can be, be about, be at least, be at least about, be at most, or be at most about, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or a number or a range between any two of these values. For example, at least 90% of the binding areas comprise no more than one nucleic acid template.

Clonal populations of no more than one target nucleic acid can be generated on or at the binding areas. The percentage of the plurality of binding areas comprising a clonal population can be different in different embodiments. In some embodiments, the percentage of the plurality of binding areas each comprising a clonal population can be, be about, be at least, be at least about, be at most, or be at most about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or a number or a range between any two of these values. For example, at least 90% of the binding areas comprise clonal populations of no more than one target sequence.

The ensemble of amplified molecules on the binding areas are different across the binding areas such that the ensemble of amplified molecules on a given binding area is different from the ensemble of amplified molecules on any other binding area of the plurality of binding areas. For example, a first binding area of the plurality of binding areas can comprise an ensemble of concatemers each containing multiple copies of the first nucleic acid template or a reverse complement thereof. The second binding area of the plurality of binding areas can comprise an ensemble of concatemers each containing multiple copies of the second nucleic acid template or a reverse complement thereof. The first nucleic acid template is different from the second nucleic acid template.

The number of binding areas of the plurality of binding areas with distinct template nucleic acids can be, be about, be at least, be at least about, be at most, or be at most about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or a number or a range between any two of these values. For example, at least 90% of the binding areas comprise distinct template nucleic acids with respect to one another.

The seeding and amplification of different nucleic acid templates on discrete binding areas of the plurality of binding areas can occur simultaneously. For example, a DNA library comprising a plurality of library constituents can be distributed over a surface comprising a plurality of binding areas to allow for simultaneous capture of the library constituents on different binding areas and amplification of a library constituent at a particular binding area of the surface. In some embodiments, the initial capture and amplification can occur at a rate faster (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values), than a secondary capture, such that a particular binding area is seeded by no more than one library constituent. Consequently, the nucleic acid population seeded at a particular binding area is often a homogeneous amplification of a single original library constituent, so as to facilitate sequencing the library constituents.

### Concatemer Strands

The first strand concatemers, also referred to as the oligonucleotide A primed concatemers, contain multiple sequence units each being complementary to the template nucleic acid. The first strand concatemers can be generated by extending oligonucleotide A along, for example, the nucleic acid template. The second strand concatemers, also referred to as the oligonucleotide B primed concatemers, contain multiple sequence units each being substantially identical to the template nucleic acid. The second strand concatemer can be generated by extending oligonucleotide B along the first strand concatemers. The second strand concatemers are reverse complementary to the first strand concatemers. In some embodiments, the second strand concatemers can also anneal to oligonucleotide A, generating concatemeric strands reverse complementary to the second strand concatemers and therefore substantially identical to the first strand concatemers. Accordingly, the first strand concatemers can also be generated by extending oligonucleotide A along the second strand concatemers. The number of first strand concatemers in a binding area can be the same as or different from the number of second strand concatemers in the same binding area. In some embodiments, the number of the first strand concatemers in a binding area outnumbers the second strand concatemers in the same binding area. In some embodiments, the number of the second strand concatemers in a binding area outnumbers the first strand concatemers in the same binding area.

A concatemer strand in a cluster can include multiple copies of a sequence unit linked in series. For example, a concatemer strand can include about at least, at least about, at most, at most about 2, 10, 25, 100 or more sequence units. The number of sequence units in a concatemer that is produced by RCA is a function of the number of times a polymerase completes a lap around a circular template during replication. The content of each sequence unit in a concatemer is substantially identical to or reverse complementary of the content of the circular template that is replicated. In some embodiments, a concatemer has an incomplete sequence unit. The concatemers in a nucleic acid cluster generated in a same binding area need not be the same length (e.g., need not have the same number of sequence units). For example, two of the first strand concatemers and/or the second strand concatemers can be the same length or different length.

The number of concatemer strands on a binding area can depend on or be similar to the number of oligonucleotide A and B attached to the binding area. A binding area of a plurality of binding area comprising at least one pair of oligonucleotide A and oligonucleotide B can contain at least two concatemer strands (e.g., a first strand and a second strand) each tethered to the binding area via oligonucleotide A or oligonucleotide B. For example, in some embodiments, a binding area can contain at least one first strand concatemer and at least one second strand concatemer. In some particular configurations (e.g., a binding area attached with a single oligonucleotide A and a single oligonucleotide B), a binding area can contain a single first strand concatemer and a single second strand concatemer.

In some embodiments, the number of concatemer strands on a binding area can be, be about, be at least, be at least about, be at most, or be at most about, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, or a number or a range between any two of these values.

In some embodiments, the concatemeric strands in a particular binding area contain the same target sequence or a reverse complement thereof, thus forming a colony which can be subjected to next generation sequencing.

### Oligonucleotide Primers

Primers used in the disclosed methods, systems and compositions are oligonucleotides having sequence complementary to portions of a template nucleic acid. Each binding area of a plurality of binding areas on a surface is attached with a plurality of first oligonucleotides (e.g., oligonucleotide A or primer A) and a plurality of second oligonucleotides (e.g., oligonucleotide B or primer A). The binding areas in a population of binding areas can have common primers (oligonucleotide A and B) when compared to one another. For example, a population of binding areas can be attached with universal primers such that the same primer sequence is present on a plurality of binding areas in the population.

Oligonucleotide A can comprise at least one capture sequence. For example, oligonucleotide A can comprise a capture sequence that is substantially complementary to the first sequence and the second sequence of the nucleic acid template. In some embodiments, oligonucleotide A can comprise two capture sequences: a first capture sequence being substantially complementary to the first primer binding sequence (e.g. the first sequence) of the nucleic acid template and a second capture sequence being substantially complementary to the second primer binding sequence (e.g., the second sequence) of the nucleic acid template. The first capture sequence and the second capture sequence can be adjacent to one another with no interval sequence in between. In some embodiments, the first capture sequence and the second capture sequence can have an interval sequence of one nucleotide, two nucleotides, three nucleotides, or more nucleotides. The first capture sequence can be 5′ of the second capture on oligonucleotide A. In some embodiments, the second capture sequence can be 5′ of the first capture sequence on oligonucleotide A. For example, for primer A shown in FIG. 2, sequence 101′ can be adjacent to sequence 102′ with no interval sequence in between, or the 3′ end of sequence 101′ can be one nucleotide, two nucleotides, three nucleotides, or more nucleotides away from 5′ end of sequence 102′. Sequence 101′ is complementary to the first sequence 101 of FIG. 1 and sequence 102′ is complementary to the second sequence 102 of FIG. 1.

Oligonucleotide B can comprise a sequence substantially identical to the third sequence of the nucleic acid template. The term “substantially identical” indicates a sequence identity with respect to another sequence of about, at least, or at least about, 70%, 75%, 80%, 85%, 90%, 95%, or a number or a range between any two of these values. In some embodiments, oligonucleotide B can comprise a sequence having a sequence identity of 100% to the third sequence of the nucleic acid template. As used herein, “sequence identity” or “identity” in the context of two nucleic acid sequences makes reference to the nucleotide bases in the two sequences that are the same when aligned for maximum correspondence over a specified comparison window using any suitable sequence alignment algorithms.

In some embodiments, the template nucleic acid is a library constituent (linear or circular) having two adapter regions (e.g., a 5′ and a 3′ adapter), and the sequence of oligonucleotide A and oligonucleotide B can be designed according to the sequence of the 5′ and 3′ adapter of the linear library constituent or a portion thereof such that oligonucleotide A is complementary to the 5′ and 3′ adapter or a portion thereof and oligonucleotide B is substantially identical to a portion of the 5′ adapter or a portion of the 3′ adapter (see e.g., in FIG. 1)

Because of the sequence complementarity, oligonucleotide A can bind to a portion of the nucleic acid template (e.g., the first sequence and the second sequence of the nucleic acid template) to generate first strand concatemers in the presence of a polymerase by extension of oligonucleotide A bound to the nucleic acid template. Oligonucleotide B can then bind to a portion of the generated first strand concatemers, generating second strand concatemers that are reverse complementary to the first strand concatemers by primer extension. Therefore, oligonucleotide A and oligonucleotide B can function as a capture primer and an amplification primer. In some embodiments, oligonucleotide A and oligonucleotide B are not reverse complementary to one another such that they do not form dimers on the surface even when coming into contact with one another.

In the embodiments herein described, a binding area of a plurality of binding areas is attached with at least one oligonucleotide A and at least one oligonucleotide B. For example, a binding area can be attached with a single oligonucleotide A and a single oligonucleotide B. In some embodiments, a binding area can be attached with multiple oligonucleotide A and oligonucleotide B. For example, a binding area can be attached with 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, or a number or a range between any two of these values, oligonucleotide A and/or oligonucleotide B.

The density of oligonucleotide A and that of oligonucleotide B on a binding area can be different or the same in different embodiments. In some embodiments, each of the density of oligonucleotide A and the density of oligonucleotide B on a binding area (e.g., a pad) can independently be, be about, be at least, be at least about, be at most, or be at most about, 1×10¹⁰, 2×10¹⁰, 3×10¹⁰, 4'10¹⁰b, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, or a number or a range between any two of these values, primers per m².

Various separation distances or average separation distances between two adjacent primers on a binding area are contemplated herein. The separation distance or average separation distance between two adjacent primers on a binding area can be, be about, be at least, be at least about, be at most, or be at most about, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, or a number or a range between any two of these values.

The number of oligonucleotide A attached to a binding area can be, and need not to be, the same as the number of oligonucleotide B attached to (e.g., immobilized to) the same binding area. Various ratios of the number of oligonucleotide A and the number of oligonucleotide B are contemplated herein. The ratio of the number of oligonucleotide A and the number of oligonucleotide B can be, be about, be at least, be at least about, be at most, or be at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. In some embodiments, the oligonucleotides A and B are provided in a plurality of primer sets, each primer set containing an oligonucleotide A and an oligonucleotide B.

Oligonucleotide A and oligonucleotide B used herein can have an identical length or different lengths. The length of an oligonucleotide primer (or two of more primers of a type or population, or each primer of a type or population) can be, be about, be at least, be at least about, be at most, be at most about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 300, 400, 500, 600, 700, 800, 900, 1000, nucleotides in length.

Oligonucleotide A and B used herein can have one or more modified nucleotides (nucleotide analogs). A nucleotide analog is a nucleotide which contains some type of modification to the base, sugar, and/or moieties as will be understood by a skilled person. In some embodiments, modifications introduced to primers can alter certain chemical properties of the primers such as to increase the stability of primer hybridization and/or to increase binding specificity.

The plurality of oligonucleotide A and the plurality of oligonucleotide B can be attached to a binding area of a plurality of binding areas via covalent or non-covalent bonds. For example, the binding area can be covalently or non-covalently attached at or near the 5′ end (e.g., one, two, three nucleotides or more nucleotides away from the 5′ end) of oligonucleotide A or oligonucleotide B such that the 3′ end of oligonucleotide A and B is available for polymerase extension. Attachment of a nucleic acid (e.g., oligonucleotide A or B) to a binding area can be mediated by any of a variety of surface chemistries such as reaction of a carboxylate moiety or succinimidyl ester moiety on the binding area with an amine-modified nucleic acid, reaction of an alkylating reagent (e.g. iodoacetamide or maleimide) on the binding area with a thiol-modified nucleic acid, reaction of an epoxysilane or isothiocyanate modified binding area with an amine-modified nucleic acid, reaction of an aminophenyl or aminopropyl modified binding area with a succinylated nucleic acid, reaction of an aldehyde or epoxide modified binding area with a hydrazide-modified nucleic acid or reaction of a thiol modified binding area with a thiol modified nucleic acid. The members of the preceding reactive pairs can be switched with regard to being present on the binding area or the nucleic acid. Click chemistry can be useful for attaching nucleic acids to a surface area. Exemplary reagents and methods for click chemistry are set forth in U.S. Pat. Nos. 6,737,236; 7,375,234; 7,427,678 and 7,763,736, each of which is incorporated herein by reference.

### Template Nucleic Acids

The template nucleic acid to be seeded and amplified can comprise primer binding sequences (e.g., a first sequence, a second sequence, a third sequence) as well as a target region containing a target sequence. The template nucleic acid can be double-stranded or single-stranded.

In some embodiments, the template nucleic acid is a circular nucleic acid (e.g. a circular DNA template) comprising a first primer binding region (e.g., formed by a first sequence and a second sequence) and a second primer binding region (e.g., formed by a third sequence). The first sequence and the second sequence can be adjacent to one another with no interval sequence in between, or the 3′ end of one sequence can be one nucleotide, two nucleotides, three nucleotides, or more nucleotides away from the 5′ end of the other sequence. The first and the second primer binding region as well as the target region can be in any configuration that enables the complementary binding between the first and second primer binding region of the template nucleic acid and oligonucleotide A and B.

For example, at least one of the first and second primer binding region of the nucleic acid template or a portion thereof can be substantially complementary to a capture sequence in oligonucleotide A while the other primer binding region of the nucleic acid template or a portion thereof can be substantially identical to a sequence in oligonucleotide B.

In some embodiments, a template nucleic acid can be a linear nucleic acid with a target sequence flanked by two primer binding regions (e.g. adapter A and adapter B shown in FIG. 1) at opposite ends of the target sequence (e.g. 3′ end and the 5′ end of the target sequence). Each of the primer binding region comprises at least one of the first sequence, the second sequence, and the third sequence. For example, one of the first sequence, the second sequence, and the third sequence can be at one end of the target sequence while the other two sequences are at the other end of the target sequence. The first sequence can be in the primer binding region at the 3′ end of the target nucleic acid while the second sequence and the third sequence are in the primer binding region at the 5′ end of the target nucleic acid. The third sequence can be at the 3′ of the second sequence. Similarly, the second sequence can be in the primer binding region at the 3′ end of the target nucleic acid while the first sequence and the third sequence are in the primer binding region at the 5′ end of the target nucleic acid. The third sequence can be at the 3′ of the first sequence. In some embodiments, the first sequence and the third sequence are in the primer binding region at the 3′ end of the target nucleic acid while the second sequence is in the primer binding region at the 5′ end of the target nucleic acid. The third sequence can be at the 5′ end of the first sequence. Similarly, the second sequence and the third sequence are in the primer binding region at the 3′ end of the target nucleic acid while the first sequence is in the primer binding region at the 5′ end of the target nucleic acid. The third sequence can be at the 5′ end of the second sequence.

The linear nucleic acid template can then be circularized to form a circular nucleic acid template (e.g., FIG. 1). A variety of methods can be used to prepare a circular template nucleic acid from a linear nucleic acid template for a RCA. In some embodiments, the circularization of the linear nucleic acid template can be produced by an enzymatic reaction, for example, by incubation with a ligation enzyme (e.g., a DNA ligase). The terminal ends of the linear nucleic acid template can be hybridized to a nucleic acid sequence (e.g. oligonucleotide A) such that the terminal ends come in close proximity (see e.g., FIG. 1). Incubating with a ligation enzyme can then result in the circularization of the hybridized linear nucleic acid template to generate a circular nucleic acid template. The circularization of the linear nucleic acid template brings together the first sequence, the second sequence and the third sequence such that in some embodiments the first sequence 101, the second sequence 102 and the third sequence 103 are adjacent to one another after circularization as shown in FIG. 1. The first, second and third sequence can be adjacent to one another with no interval sequence in between, or the 3′ end of one sequence can be one nucleotide, two nucleotides, three nucleotides, or more nucleotides away from the 5′ end of another sequence.

In some embodiments, the linear nucleic acid template molecule can be a linear library constituent having a distinct 5′ and 3′ adapter region flanking a target region (e.g., in FIG. 1). The adapter regions can have any of a variety functions including, for example, providing a binding site that complements a capture primer (e.g. oligonucleotide A attached to a binding area), providing a primer binding site for replicating the circular template, providing a primer binding site for replicating a complement of the circular template, providing a tag that is associated with the target region (e.g. a tag indicating the source of the target region such as a barcode or a tag used for identifying errors introduced during amplification of the target region etc.). The adapter regions or portions thereof can be common to a population of circular templates or to a population of concatemers. Whether or not the adapter regions have common sequences, the target regions in the population of circular templates or in the population of concatemers can have different sequences (e.g. different target sequences). Thus, when comparing sequence units between two or more concatemers, or between two or more circular templates, the sequence units can have common sequence regions (e.g. universal primer binding sites) and/or the sequence units can have regions of differing sequence (e.g. different target sequences).

The linear library constituent can be annealed to the surface bound oligonucleotide A having regions complementary to the 5′ and 3′ adapters of the linear library constituent or a portion thereof, and oriented so as to position the 5′ and 3′ ends in close proximity. The 5′ and 3′ ends of the linear library constituent are ligated so as to form a circular library constituent. In some embodiments, the nucleic acid template can be a circular library constituent (e.g. a DNA library ring).

The methods herein described can accommodate library constituent of any length. For example, the library constituents used herein can have lengths of less than 50, 45, 40, 35, 30, 25, 20 or less than 20, or alternately at least 300, 400, 500, 600, 700, 800, 900, 1000, 2000, 3000, or greater than 3000 bases.

The template nucleic acids used in the methods, compositions, and systems set forth herein can be DNA such as genomic DNA, synthetic DNA, amplified DNA, complementary DNA (cDNA) or the like. The template nucleic acids used herein can also be RNA such as mRNA, ribosomal RNA, tRNA or the like. The template nucleic acids used herein can also comprise nucleic acid analogs comprising modifications to the phosphate moiety, the sugar moiety and/or the nitrogenous base of a nucleotide analog.

The length of the target region in a template nucleic acid can be selected to suit a particular application of the methods described herein. For example, the length can be can be about, at least, at least about, at most or at most about 50, 100, 250, 500, 1000, 1×10⁴, 1×10⁵ or more nucleotides.

The target nucleic acids used herein can be derived from a biological source, synthetic source or amplification product. Exemplary organisms from which nucleic acids can be derived include, for example, a mammal such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as pneumocystis carini , Takifugu rubripes, yeast, Saccharamoyces cerevisiae or Schizosaccharomyces pombe; or a plasmodium falciparum. Nucleic acids can also be derived from a prokaryote such as a bacterium, Escherichia coli, staphylococci or mycoplasma pneumoniae; an archae; a virus such as Hepatitis C virus, influenza virus, corona virus or human immunodeficiency virus; or a viroid. Nucleic acids can be derived from a homogeneous culture or population of the above organisms or from a collection of several different organisms, for example, in a community or ecosystem. Nucleic acids can be isolated using methods known in the art including, for example, those described in Sambrook et al., Molecular Cloning: A Laboratory Manual,3rd edition, Cold Spring Harbor Laboratory, New York (2001) or in Ausubel et al., Current Protocols in Molecular Biology, John Wiley and Sons, Baltimore, Md. (1998), each of which is incorporated herein by reference.

### Surface and Binding Area

In the methods, compositions and systems herein described, a surface is provided, such as a flow cell surface, the surface comprising one or a plurality of binding areas. The binding areas can be used, for example, for seeding and amplifying the nucleic acid templates according to the methods disclosed herein. The flow cell surface can be generated using any of a variety of suitable methods. For example, the flow cell surface can be generated using top-down lithography or bottom-up self-assembly of particles described in U.S. Provisional Application No. 63/137,064, entitled “Surface Structuring With Colloidal Assembly” filed on Jan. 13, 2021, the content of which is incorporated herein by reference in its entirety.

The surface used herein can be generated using colloidal self-assembly (bottom-up) of particles. For example, the surface used herein can be made by providing a structure subsumed in a liquid, delivering a plurality of particles (e.g. beads) to a surface of the liquid. The surface of the liquid can be the top surface of the liquid which is in contact with another medium, such as air. A percentage of the plurality of particles (e.g., 5%, 10%, 20%, 25%, 30%, 35%, 40%, 45%, 50%, 55%, 60%, 65%, 70%, 75%, 80%, 85%, 90%, 95%, or a number or a range between any two of these values, or more of the plurality of particles) can self-assemble into closely packed, ordered particles. The method can include removing the liquid between the particles and the planar structure, such that the plurality of particles comes into contact with the planar structure. The plurality of particles on the surface of the liquid and/or in contact with the planar structure can specify a plurality of binding areas on the planar structure.

The surface used herein can also be made, for example, by providing a planar structure having deposited thereon an active site layer (or a substance layer or a binding site layer) and a masking layer. The method can include depositing a plurality of particles (e.g. beads) onto the masking layer of the planar structure. The method can include exposing the planar structure to an etching agent at action so as to differentially remove the masking layer from regions not shielded by the plurality of beads. The method can include removing the masking layer and the active site layer from regions not shielded from the etching layer by the plurality of beads. The method can include removing remaining masking layer from regions shielded by the plurality of beads with the active site layer at the binding areas remaining. The method thereby specifies a plurality of binding areas comprising the remaining active site layer.

The number of binding areas on a flow cell surface can be, be about, be at least, be at least about, be at most, or be at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, or a number or a range between any two of these values. In some embodiments, the surface comprises about 10⁴ binding areas to about 10⁸ binding areas. In some embodiments, the surface comprises a plurality of binding areas of at least 10,000 binding areas. Each of the plurality of binding areas can correspond to a variety of different forms and shapes such as circular, round, oval, rectangular, or square. Each of the plurality of binding areas can have a center point and a diameter. The binding areas on a flow cell surface can be ordered, well packed, and/or can have high density. The positions (or locations) of the binding areas on the flow cell surface can be random and non-predetermined. In some instances, the flow cell surface includes a plurality of binding areas of at least 10,000 ordered, well packed, and/or high density binding areas separated by disjunctions (e.g., 5, 10, 15, 20, 25, 30, 35, 40, 45, 50, 55, 60, 65, 70, 75, 80, 85, 90, 95, 100, or a number or a range between any two of these values, or more disjunctions). The configurations of the ordered binding areas and/or the disjunctions can be non-predetermined and/or can be randomly distributed.

The binding areas can contemplate different separations or pitches between any two neighboring binding areas. In some embodiments, the pitch between any binding area and any nearest neighbor binding area, measured from the center of the first binding area to the center of the nearest neighbor binding area, can be at least twice as large as the diameter of the first binding area. For example, the pitch between any binding area and any nearest neighbor binding area can about 1 nm to about 100 μm. Separation between any binding area and any nearest neighbor binding area, measured from an edge of the first binding area to a center of the nearest neighbor binding area is at least twice as large as the diameter of the first binding area. The two edges are closer than (or at least as close as) the distance between any other edge of the first binding area and any edge of the second binding rea. The size (e.g. radius, diameter, width or height) of the plurality of binding areas can be about 1 nm to about 100 μm.

The plurality of binding areas or a percentage of the binding areas can share a common pattern (e.g. shape or dimension) or a different pattern. In some embodiments, the plurality of binding areas comprises unpatterned binding areas. The plurality of binding areas or a percentage of the binding areas can be randomly positioned on the flow cell surface or can be arrayed on the flow cell in a predetermined set of locations. In some embodiments, the plurality of binding areas is arrayed so as to form a first region having regularly positioned (or ordered) binding areas and a second region having randomly (or irregularly) positioned binding areas.

The binding areas on the surface can be functionalized to facilitate the attachment of primers. For example, the binding areas can comprise a carboxylate moiety or succinimidyl ester moiety to attach an amine-modified primer, an alkylating reagent (e.g. iodoacetamide or maleimide) to attach a thiol-modified primer, an epoxysilane or isothiocyanate to attach an amine-modified primer, an aminophenyl or aminopropyl moiety to attach a succinylated primer, an aldehyde or epoxide moiety to attach hydrazide-modified primer, or a thiol group to attach a thiol modified primer. The members of the preceding reactive pairs can be switched with regard to being present on the binding area or the primer. Click chemistry can be useful for attaching nucleic acids to a surface area. Exemplary reagents and methods for click chemistry are set forth in U.S. Pat. Nos. 6,737,236; 7,375,234; 7,427,678 and 7,763,736, each of which is incorporated herein by reference. Methods for functionalizing binding areas of a flow cell surface is also described, in U.S. Provisional Application No. 63/137,064, entitled “Surface Structuring With Colloidal Assembly” filed on Jan. 13, 2021, the content of which is incorporated herein by reference.

### Polymerase

Any of a variety of polymerases can be used in a method or composition set forth herein, for example, to form a polymerase-nucleic acid complex or to carry out primer extension. Polymerases that can be used include naturally occurring polymerases and modified variations thereof, including, but not limited to, mutants, recombinants, fusions, genetic modifications, chemical modifications, synthetics, and analogs. Naturally occurring polymerases and modified variations thereof are not limited to polymerases that have the ability to catalyze a polymerization reaction. The naturally occurring and/or modified variations thereof can, for example, have the ability to catalyze a polymerization reaction in at least one condition that is not used during formation or examination of a stabilized ternary complex. The naturally occurring and/or modified variations that participate in polymerase-nucleic acid complexes can, for example, have modified properties, for example, enhanced binding affinity to nucleic acids, reduced binding affinity to nucleic acids, enhanced binding affinity to nucleotides, reduced binding affinity to nucleotides, enhanced specificity for next correct nucleotides, reduced specificity for next correct nucleotides, reduced catalysis rates, catalytic inactivity etc. Mutant polymerases include, for example, polymerases wherein one or more amino acids are replaced with other amino acids, or insertions or deletions of one or more amino acids. Exemplary polymerase mutants that can be used to form a stabilized ternary complex include, for example, those set forth in U.S. Patent Application Publication No. 2020/0087637 and U.S. Pat. Nos. 10,584,379 and 10,597,643, each of which is incorporated herein by reference. In some embodiments, the polymerases used herein have strand-displacement activity alone or in combination with a strand displacement factor such as a helicase.

Polymerases used herein can be attached with an exogenous label moiety (e.g. an exogenous fluorophore), which can be used to detect the polymerase. The label moiety can, for example, be attached after the polymerase has been at least partially purified using protein isolation techniques. For example, the exogenous label moiety can be covalently linked to the polymerase using a free sulfhydryl or a free amine moiety of the polymerase. This can involve covalent linkage to the polymerase through the side chain of a cysteine residue, or through the free amino moiety of the N-terminus. An exogenous label moiety can also be attached to a polymerase via protein fusion. Exemplary label moieties that can be attached via protein fusion include, for example, green fluorescent protein (GFP), phycobiliproteins (e.g., phycocyanin and phycoerythrin) or wavelength-shifted variants of GFP or phycobiliproteins. In some embodiments, a polymerase used herein need not be attached to an exogenous label.

Different activities of polymerases can be exploited in a method set forth herein. A polymerase can be useful, for example, in RCA amplification (e.g. in a primer extension step), or in nucleic acid sequencing. Polymerase can be obtained from a variety of known sources and applied in accordance with the teachings set forth herein and recognized activities of polymerases. The polymerases can be DNA polymerases, RNA polymerase, or other types of polymerases such as reverse transcriptase.

Exemplary DNA polymerases include, but are not limited to, bacterial DNA polymerases, eukaryotic DNA polymerases, archaeal DNA polymerases, viral DNA polymerases and phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, €, η, ζ, λ, σ, μ, and k, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cpl DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2. Engineered and modified polymerases also are useful in connection with the disclosed techniques. For example, modified versions of the extremely thermophilic marine archaea Thermococcus species 9° N (e.g., Therminator DNA polymerase from New England BioLabs Inc.; Ipswich, Mass.) can be used.

Exemplary RNA polymerases include, but are not limited to, viral RNA polymerases such as T7 RNA polymerase, T3 polymerase, SP6 polymerase, and K11 polymerase; Eukaryotic RNA polymerases such as RNA polymerase I, RNA polymerase II, RNA polymerase III, RNA polymerase IV, and RNA polymerase V; and Archaea RNA polymerase.

Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, and Telomerase reverse transcriptase that maintains the telomeres of eukaryotic chromosomes.

### Applications in Sequencing

The amplified nucleic acids generated on the binding areas of a flow cell surface can be The methods disclosed herein can be used in various sequencing platforms, including but not limited to, sequencing-by-synthesis or sequencing-by-binding (sometimes collectively referred to as sequencing-by-incorporation chemistries), pH-based sequencing, sequencing by polymerase monitoring, sequencing by hybridization, and other methods of massively parallel sequencing or next-generation sequencing. In some embodiments, the sequencing is carried out as described in U.S. Pat. No. 10,077,470, which is incorporated by reference herein in its entirety. Suitable surfaces for carrying out sequencing include, but are not limited to, a planar substrate, a hydrogel, a nanohole array, a microparticle, or nanoparticle.

### Sequencing-By-Binding

The methods, compositions and systems disclosed herein for performing RCA can be used in sequencing-by-binding (SBB) methods, compositions and systems.

Sequencing by binding has been described, for example, in U.S. Pat. Nos. 10,443,098 and 10,246,744, and US Pat. App. Pub. No. 2018,0044727; the content of each is incorporated herein by reference in its entirety. In SBB, the polymerase undergoes conformational transitions between open and closed conformations during discrete steps of a reaction. In one step, the polymerase binds to a primed template nucleic acid to form a binary complex, also referred to herein as the pre-insertion conformation. In a subsequent step, an incoming nucleotide is bound and the polymerase fingers close, forming a pre-chemistry conformation comprising the polymerase, a primed template nucleic acid and a nucleotide, wherein the bound nucleotide has not been incorporated. This step is also referred to as an examination step. The nucleotide can be labeled or unlabeled. Likewise, the polymerase can be labeled or unlabeled. The examination step can involve providing a primed template nucleic acid and contacting the primed template nucleic acid with a polymerase (e.g. a DNA polymerase) and one or more test nucleotides being investigated as the possible next correct nucleotide. The polymerase configuration and/or interaction with the primed template nucleic acid and further with a nucleotide can be monitored during an examination step to identify the next correct base in the template nucleic acid. Accordingly, in some embodiments, the SBB procedure includes a monitoring step that monitors or measures the interaction between the polymerase and the primed template nucleic acid in the presence of the test nucleotides. In some embodiments, the examination step determines the identity of the next correct nucleotide without requiring incorporation of that nucleotide (e.g. either without, or before chemical linkage of that nucleotide to the 3′-end of the primer through a covalent bond). For example, the primer of the primed template nucleic acid molecule can include a blocking group that precludes enzymatic incorporation of an incoming nucleotide into the primer. The reaction mixture used in the examination step can, for example, comprise catalytic metal ions at a low or deficient level to prevent the chemical incorporation of the nucleotide into the primer of the primed template nucleic acid. In some embodiments, the reaction mixture used in the examination step comprises a stabilizer that stabilize ternary complexes while precluding incorporation of any nucleotide into the primer, such as a non-catalytic metal ion that inhibits polymerization.

Generally, an examination step involves binding a polymerase to the polymerization initiation site of a primed template nucleic acid in a reaction mixture comprising one or more nucleotides, and monitoring the interaction. An examination step typically includes the following substeps: (1) providing a primed template nucleic acid (i.e., a template nucleic acid molecule hybridized with a primer that optionally can be blocked from extension at its 3′ end); (2) contacting the primed template nucleic acid with a reaction mixture that includes a polymerase and at least one nucleotide; (3) monitoring the interaction of the polymerase with the primed template nucleic acid molecule in the presence of the nucleotide(s) and without chemical incorporation of any nucleotide into the primed template nucleic acid; and (4) determining from the monitored interaction the identity of the next base in the template nucleic acid (i.e., the next correct nucleotide). Examination typically involves detecting polymerase interaction with a template nucleic acid. Detection can include optical, electrical, thermal, acoustic, chemical and mechanical means. The examination step of the sequencing reaction can be repeated, in some embodiments, 1, 2, 3, 4 or more times prior to the optional incorporation step.

In SBS, a reaction mixture used in the examination step can include 1, 2, 3, or 4 types of nucleotide molecules. The nucleotides can be selected from dATP, dTTP (or dUTP), dCTP, and dGTP. The examination reaction mixture can comprise one or more triphosphate nucleotides and one or more diphosphate nucleotides. A ternary complex can form between the primed template nucleic acid, the polymerase, and any one of the four nucleotide molecules so that four types of ternary complexes can be formed.

An incorporation step can be concurrent with or separate from the examination step. In some embodiments of a SBB procedure, the examination step is followed by an incorporation step that adds one or more complementary nucleotides to the 3′ end of the primer component of the primed template nucleic acid. The polymerase, primed template nucleic acid and newly incorporated nucleotide produce a post-chemistry conformation. Both pre-chemistry conformation and the post-chemistry conformation can be referred to as a ternary complex, each comprising a polymerase, a primed template nucleic acid and a nucleotide, wherein the polymerase is in a closed state and facilitates interaction between a next correct nucleotide and the primed template nucleic acid. During the incorporation step, divalent catalytic metal ions, such as Mg²⁺, mediate a chemical step involving nucleophilic displacement of a pyrophosphate (PPi) by the 3′-hydroxyl of the primer terminus. The polymerase returns to an open state upon the release of PPi.

The incorporation step can be facilitated by an incorporation reaction mixture. The incorporation reaction mixture can have a different composition of nucleotides than the examination reaction. For example, the examination reaction can include one type of nucleotide and the incorporation reaction can include another type of nucleotide. By way of another example, the examination reaction comprises one type of nucleotide and the incorporation reaction comprises four types of nucleotides, or vice versa. The examination reaction mixture can be altered or replaced by the incorporation reaction mixture.

In some embodiments of the SBB procedure, an examination step is followed by removal of the labeled nucleotide without being incorporated, and is then followed by de-blocking of the 3′ end of the primer (or extended primer) of the primed template nucleic acid so as to render it suitable for extension. Unlabeled, 3′ blocked nucleotides are then added, followed by a chemical incorporation step wherein a phosphodiester bond is formed with concomitant pyrophosphate cleavage from the nucleotide (nucleotide incorporation), to form an extension strand that has been extended by one base and that is not competent for further extension without modification. Unincorporated blocked extension bases are removed and labeled bases added, so that they can from ternary complexes at positions where they base pair with the template. These ternary complexes are assayed for fluorescence or other output to determine the identity of the paired base, and then the process is repeated through removal of the labeled base, chemical modification of the extending strand to reveal a 3′OH, and contacting with a population of 3′blocked, unlabeled nucleotides for another single base extension.

### Sequencing-By-Synthesis

The methods, compositions and systems disclosed herein for performing RCA can be used in sequencing-by-synthesis (SBS) methods, compositions and systems.

SBS generally involves the enzymatic extension of a nascent primer through the iterative addition of nucleotides against a template strand to which the primer is hybridized. SBS differs from SBB, above, in that labeled nucleotides are incorporated into the extending strand, assayed and then the label is removed or deactivated, and the 3′ block removed, to iteratively sequence a template. In SBB, a labeled base is not incorporated into an extending strand. Rather, ternary complex formation is assayed, usually for the presence of a labeled base but sometimes for the presence of a labeled polymerase or other feature, after which point the complex is disassembled and a 3′ blocked, unlabeled base is used to extend the primer strand. Briefly, SBS can be initiated by contacting target nucleic acids, attached to sites in a flow cell, with one or more labeled nucleotides, DNA polymerase, etc. Those sites where a primer is extended using the target nucleic acid as template will incorporate a labeled nucleotide that can be detected. Detection can include scanning using an apparatus or method set forth herein. The labeled nucleotides can, for example, further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the vessel (before or after detection occurs). Washes can be carried out between the various delivery steps. The cycle can be performed n times to extend the primer by n nucleotides, thereby detecting a sequence of length n. Exemplary SBS procedures, reagents and detection components that can be readily adapted for use with a method, system or apparatus of the present disclosure are described, for example, in Bentley et al., Nature 456:53-59 (2008), WO 04/018497; WO 91/06678; WO 07/123744; U.S. Pat. Nos. 7,057,026; 7,329,492; 7,211,414; 7,315,019 or 7,405,281, and US Pat. App. Pub. No. 2008/0108082 A1, each of which is incorporated herein by reference.

### Systems

Systems disclosed herein for nucleic acid amplification, detection and/or sequencing can include a vessel, solid support or other apparatus for carrying out a nucleic acid amplification, detection and/or sequencing. For example, the system can include an array, flow cell, multi-well plate, test tube, channel in a substrate, collection of droplets or vesicles, tray, centrifuge tube, tubing or other convenient apparatus. The apparatus can be removable, thereby allowing it to be placed into or removed from the system. As such, a system can be configured to process a plurality of apparatus (e.g. vessels or solid supports) sequentially or in parallel. The system can include a fluidic component configured to deliver one or more reagents (e.g., one or more reagents in a solution) to a vessel or solid support, for example, via channels or droplet transfer apparatus (e.g. electrowetting apparatus). Any of a variety of detection apparatus can be configured to detect the vessel or solid support where reagents interact. Exemplary systems having fluidic and detection components those set forth in US Pat. App. Pub. No. 2018/0280975A1; U.S. Pat. Nos. 8,241,573; 7,329,860 or 8,039,817; or US Pat. App. Pub. Nos. 2009/0272914 Al or 2012/0270305 A1, each of which is incorporated herein by reference.

In some embodiments, the system for nucleic acid amplification comprises a flow cell having distributed thereon a plurality of binding areas, each binding area attached thereto a plurality of oligonucleotide A and a plurality of oligonucleotide B herein described. The oligonucleotide A is designed to be complementary to a portion of a template nucleic acid to be amplified, while the oligonucleotide B is designed to be substantially identical to a different portion of the template nucleic acid. In some embodiments, the plurality of binding areas or a population of binding areas can be attached with universal oligonucleotide primers such that the same oligonucleotide A and oligonucleotide B are presented on the binding areas.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g.,“a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of nucleic acid amplification, comprising
(a) providing a first circular DNA template comprising a first sequence, a second sequence, and a third sequence;
(b) providing a surface comprising a plurality of binding areas, wherein each of the plurality of binding areas is attached thereto a plurality of oligonucleotide A and a plurality of oligonucleotide B, wherein the oligonucleotide A comprises a first capture sequence that is complementary to the first sequence and a second capture sequence that is complementary to the second sequence, and wherein the oligonucleotide B comprises the third sequence; and
(c) contacting the first circular DNA template with the plurality of oligonucleotide A attached to a first binding area of the plurality of binding areas in the presence of a DNA polymerase to generate amplified single stranded concatemers of the first DNA template via rolling circle amplification (RCA), and contacting the single stranded oligonucleotide A primed concatemers to the plurality of oligonucleotide B to produce complementary concatemers of the first DNA template.

2. The method of claim 1, further comprising providing a second circular DNA template comprising the first sequence, the second sequence, and the third sequence; and contacting the second circular DNA template with the plurality of oligonucleotide A being attached to a second binding area of the plurality of binding areas in the presence of the DNA polymerase to generate amplified single stranded concatemers of the second DNA template via RCA, and contacting the single stranded oligonucleotide A primed concatemers of the second DNA template to the plurality of oligonucleotide B to generate complementary concatemers of the second DNA template.

3. The method of claim 1 or 2, wherein contacting the first circular DNA template with the plurality of oligonucleotide A attached to a first binding area of the plurality of binding areas in the presence of a DNA polymerase and contacting the second circular DNA template with the plurality of oligonucleotide A being attached to a second binding area of the plurality of binding areas in the presence of the DNA polymerase occur simultaneously.

4. The method of any one of claims 1-3, wherein contacting the first circular DNA template with the plurality of oligonucleotide A attached to the first binding area of the plurality of binding areas in the presence of the DNA polymerase to generate amplified single stranded concatemers of the first DNA template via RCA comprises:
hybridizing the first circular DNA template with an oligonucleotide A attached to the first binding area of the plurality of binding areas; and
extending the oligonucleotide A bound to the first circular DNA template along the first circular DNA template by the DNA polymerase, whereby generating an amplified single stranded concatemer of the first DNA template.

5. The method of any one of claims 1-4, wherein contacting the single stranded oligonucleotide A primed concatemers to the plurality of oligonucleotide B to produce complementary concatemers of the first DNA template comprises:
hybridizing a single stranded oligonucleotide A primed concatemer with an oligonucleotide B attached to the first binding area of the plurality of binding areas; and
extending the oligonucleotide B bound to the single stranded oligonucleotide A primed concatemer by the DNA polymerase, whereby generating a complementary concatemer of the first DNA template.

6. The method of claim 5 or 6, wherein extending of oligonucleotide A bound to the first circular DNA template along the first circular DNA template by the DNA polymerase and extending the oligonucleotide B bound to the single stranded oligonucleotide A primed concatemer occur simultaneously.

7. The method of any one of claims 1-6, comprising contacting complementary concatemers of the first DNA template with one or more oligonucleotide A of the plurality of oligonucleotide A attached to the first binding area to produce additional concatemers of the first DNA template.

8. The method of any one of claims 2-7, comprising contacting complementary concatemers of the second DNA template with one or more oligonucleotide A of the plurality of oligonucleotide A attached to the second binding area to produce additional concatemers of the second DNA template.

9. The method of any one of claims 1-8, wherein the complementary concatemer of the first DNA template is reverse complementary to the single stranded concatemer of the first DNA template.

10. The method of any one of claims 1-9, wherein the first circular DNA template and the second circular DNA template are provided in the same sample.

11. The method of any one of claims 1-10, wherein the first circular DNA template, the second circular DNA template, or both is a single-stranded DNA.

12. The method of any one of claims 1-11, wherein the first circular DNA template, the second circular DNA template, or both is circularized from a linear nucleic acid template.

13. The method of any one of claims 1-12, wherein the surface is a flow cell surface.

14. The method of claim 13, wherein the RCA reaction is carried out within a flow cell.

15. The method of any one of claims 1-14, comprising terminating the RCA reaction by exhaustion of oligonucleotide A, oligonucleotide B, or both.

16. The method of any one of claims 1-15, wherein the method does not comprise terminating the RCA reaction by denaturing the DNA polymerase.

17. The method of any one of claims 1-15, wherein the method does not comprise terminating the RCA reaction by removing the DNA polymerase.

18. The method of any one of claims 1-17, wherein the RCA is performed at about 37° C.

19. The method of any one of claims 1-18, wherein the DNA polymerase is Phi29 DNA polymerase.

20. The method of any one of claims 1-19, wherein the first capture sequence is 5′ of the second capture sequence on the oligonucleotide A.

21. The method of any one of claims 1-19, wherein the second capture sequence is 5′ of the first capture sequence on the oligonucleotide A.

22. The method of any one of claims 1-21, wherein the plurality of oligonucleotide A, the plurality of oligonucleotide B, or both are covalently conjugated to the first binding area, the second binding area, or both.

23. The method of any one of claims 1-21, wherein the plurality of oligonucleotide A, the plurality of oligonucleotide B, or both are non-covalently attached to the first binding area, the second binding area, or both.

24. The method of any one of claims 1-23, wherein the plurality of oligonucleotide A and the plurality of oligonucleotide B are each attached at or near a 5′ end of the oligonucleotide A or oligonucleotide B.

25. The method of any one of claims 1-24, wherein the plurality of oligonucleotide A and the plurality of oligonucleotide B are not reverse complementary to one another.

26. The method of any one of claims 1-25, wherein a binding area of the plurality of binding areas is attached thereto a single oligonucleotide A, a single oligonucleotide B, or both.

27. The method of any one of claims 1-25, wherein a binding area of the plurality of binding areas is attached thereto at least 10,000 oligonucleotide A, at least 10,000 oligonucleotide B, or both.

28. The method of any one of claims 1-27, wherein a ratio of the plurality of oligonucleotide A and the plurality of oligonucleotide B attached to a binding area of the plurality of binding area is about 100:1 to about 1:100.

29. The method of any one of claims 1-28, wherein the first binding area comprises a clonal population of the first DNA template.

30. The method of any one of claims 1-29, wherein the first binding area does not comprise the second DNA template.

31. The method of any one of claims 2-30, wherein the second binding area comprises a clonal population of the second DNA template.

32. The method of any one of claims 2-31, wherein the second binding area does not comprise the first DNA template.

33. The method of any one of claims 1-32, wherein at least 90% of the binding areas comprise clonal populations of no more than one nucleic acid template.

34. The method of any one of claims 1-33, wherein at least 90% of the binding areas comprise distinct template nucleic acids with respect to one another.

35. The method of any one of claims 1-34, wherein the first sequence and the second sequence are adjacent to one another.

36. The method of any one of claims 1-35, wherein the third sequence is adjacent to the first sequence or the second sequence.

37. The method of any one of claims 1-36, wherein the concatemers of the first DNA template and the complementary concatemers of the first DNA template are attached to the first binding area of the plurality of binding areas via the plurality of oligonucleotide A and the plurality of oligonucleotide B attached to the first binding area.

38. The method of any one of claims 1-37, wherein the concatemers of the second DNA template and the complementary concatemers of the second DNA template are attached to the second binding area of the plurality of binding areas via the plurality of oligonucleotide A and the plurality of oligonucleotide B attached to the second binding area.

39. The method of any one of claims 1-36, wherein the surface comprises about 10⁴ binding areas to about 10⁸ binding areas.

40. The method of any one of claims 1-37, wherein the surface comprises at least 10,000 ordered binding areas separated by disjunctions that are not predetermined and/or are randomly distributed.

41. The method of any one of claims 1-40, wherein one, one or more, or each, of the plurality of binding areas has a circular shape.

42. The method of any one of claims 1-41, wherein the size of one, one or more, or each, of the plurality of binding areas is about 10⁻⁹ m to about 10⁻⁴ m.

43. The method of claim 42, wherein the size of the one, one or more, or each, of the plurality of binding areas is a width or a radius of the binding areas.

44. The method of any one of claims 1-43, wherein the surface is a planar surface.
